(19) United States
(12) Patent Application Publication
(10) Pub. No.: US 2017/0224662 A1
(43) Pub. Date: Aug. 10, 2017

(54) Aqueous Formulations and Methods of Preparation and Use Thereof

(71) Applicant: The Medicines Company, Parsippany, NJ (US)

(72) Inventors: Rajeshwar Motheram, Dayton, NJ (US); Nitin Joshi, Redwood City, CA (US); John C.R. Randle, Brookline, MA (US); Walter Joseph Lunsmann, Harvard, MA (US)

(21) Appl. No.: 15/413,213

(22) Filed: Jan. 23, 2017

Related U.S. Application Data
(60) Provisional application No. 62/281,853, filed on Jan. 22, 2016.

(51) Int. Cl. A61K 31/4174; A61K 47/02; A61M 5/20; A61K 45/06; A61K 9/19; A61K 9/08; A61K 47/12; A61K 47/40

(52) U.S. Cl. CPC A61K 31/4174; A61K 47/12; A61K 47/02; A61K 47/40; A61K 45/06; A61K 9/19; A61K 9/08; A61M 5/2033

(57) ABSTRACT

Provided herein are stable aqueous formulations of anesthetic compounds, suitable for injection or infusion.

### CROSS-REFERENCE TO RELATED APPLICATIONS

This application claims the benefit of U.S. Provisional Application No. 62/281,853, filed on Jan. 22, 2016, the contents of which are incorporated by reference herein, in their entirety and for all purposes.

### FIELD OF THE DISCLOSURE

The disclosure relates generally to anesthetic formulations and their methods of preparation and use. More particularly, the disclosure relates to stabilized aqueous formulations of cyclopropyl-MOC-metomidate (CPMM).

### BACKGROUND

The number of surgical procedures performed has been steadily increasing. At the same time, surgical care and procedural medicine continue to move towards care with lighter anesthesia, minimal and focused procedural sedation, primarily outpatient care and management, and non-physician care providers. The highly dynamic nature of surgical and procedural intervention, as well as the often very short duration of these procedures, is better suited to potent yet rapidly reversible sedative/anesthetic agents whose pharmacokinetics (PK) and pharmacodynamics (PD) are well-matched to the procedures being performed. In general, current agents are either too long-acting and/or do not take effect quickly enough. The result is a clear unmet need for safer, more effective drugs that exhibit predictable, rapid PK/PD and allow precisely-tailored control of sedation and anesthesia. The development of newer sedative/anesthetic drugs and drug products that (i) are highly potent, (ii) have minimal hemodynamic effects or other toxicities, (iii) have PK/PD profiles that enable them to be more readily titratable and enable rapid emergence from anesthesia, and (iv) are convenient for use and well-tolerated, are highly desired to meet evolving patient and healthcare needs.

Cyclopropyl-MOC-metomidate (CPMM) is an intravenously-administered general anesthetic for monitored anesthesia care (MAC) and/or general anesthesia in patients undergoing diagnostic or therapeutic procedures.

CPMM is a potent positive allosteric modulator of the γ-aminobutyric acid Type A (GABA_{A}) receptor, which is a ligand-gated ion channel, and leads to CPMM's sedative and anesthetic effects. CPMM contains a methoxycarbonyl ester moiety that is designed to undergo rapid hydrolysis in the body by plasma and tissue esterases to produce an inactive carboxylic acid metabolite, CPM-acid. This mechanism of inactivation results in a very rapid and predictable profile for offset of action.

However, CPMM exhibits only moderate solubility in aqueous solution and the metabolically-labile ester is also somewhat unstable in aqueous solution, which can lead to degradation of the compound upon storage in an aqueous medium. Thus, a need exists for formulation of anesthetic compounds, such as CPMM, in which the anesthetic is sufficiently soluble and, additionally, has a desired level of stability.

### SUMMARY

Provided herein are formulations of CPMM or pharmaceutically acceptable salt thereof, and a solubilizer. The formulations disclosed herein are preferably sterile. The CPMM can be present in an amount of 0.1 mg/mL to 20 mg/mL, e.g., 1 to 10 mg/mL or 2 to 5 mg/mL. The formulation can have a pH in a range of 2 to 7, e.g., 3 to 7, 4 to 6.5, or 5 to 6.5. In some cases, the pH is 6 to 7.4. The formulation can optionally further comprise a buffer. The buffer can comprise one or more of NaOH, KOH, triethylamine, meglumine, diethanolamine, triethylamine, ammonium hydroxide, ammonium acetate, L-arginine, histidine, citrate buffer, a phosphate buffer, sodium bicarbonate, tris(hydroxymethyl)aminomethane), N-(2-hydroxyethyl)piperazine-N′-2-ethanesulfonic acid, acetate, citrate, ascorbate, glycine, glutamate, lactate, malate, formate, and sulfate. In some cases, the buffer comprises citrate buffer and NaOH.

In various cases, the solubilizer comprises a cyclodextrin, e.g., (β-cyclodextrin, or more specifically, sulfobutylether-β-cyclodextrin, hydroxypropyl-β-cyclodextrin, or a combination thereof. In some specific cases, the solubilizer comprises sulfobutylether-β-cyclodextrin. The solubilizer can be present in a concentration in a range of about 6% w/w to about 30% w/w, e.g., about 8% w/w to about 20% w/w, or about 8% w/w to about 12% w/w, or more specifically, about 10% w/w. The solubilizer can be present in a molar ratio to the compound (CPMM) of 1:1 to 8:1, e.g., 1:1 to 4:1. In some cases, the ratio of compound complexed to the solubilizer to uncomplexed compound is 1.5:1 to 16:1.

In certain embodiments, the formulation disclosed herein can comprise the compound at a concentration in a range of 2 mg/mL to 5 mg/mL, the solubilizer comprises hydroxypropyl-β-cyclodextrin at a concentration in a range of 8% w/v or w/w to 12% w/v or w/w, and the formulation has a pH in a range of about 5 to 7. In some embodiments, the formulation disclosed herein can comprise the compound at a concentration in a range of 2 mg/mL to 5 mg/mL, the solubilizer comprises sulfobutylether-β-cyclodextrin at a concentration in a range of 6% w/v or w/w to 12% w/v or w/w, and the formulation has a pH in a range of about 3 to 7 (e.g., 5 to 6).

The formulations disclosed herein can further comprise an antimicrobial agent. The formulations disclosed herein can have 5% or less (e.g., 1% or less) total degradants after storage at a temperature of 2° C. to 8° C. for at least 6 months, at least 12 months, or at least 24 months. The formulations disclosed herein can have 5% or less (e.g., 1% or less) total degradants after storage at frozen conditions (e.g., a temperature of −10° C. to 0° C.) for at least 12 months, or at least 24 months. The formulations disclosed herein can have 5% or less (e.g., 1% or less) total degradants after storage at room temperature (e.g., a temperature of 15° C. to 30° C.) for at least 6 months.

The formulations disclosed herein can be lyophilized. In some cases, the lyophilized formulation is provided in a kit, with the lyophilized formulation in a container and with instructions for preparing an aqueous, sterile formulation form the lyophilized formulation and a diluent (e.g., saline, sterile water, PBS, or a mixture thereof).

Further provided herein is a liquid pharmaceutical formulation consisting essentially of (1) cyclopropyl-MOC-metomidate (CPMM) or a pharmaceutically acceptable salt thereof, (2) a solubilizer, and (3) a buffer or base in an aqueous medium. The solubilizer can be P-cyclodextrin (e.g., sulfobutylether-β-cyclodextrin, hydroxypropyl-β-cyclodextrin, or a combination thereof). The buffer can comprise citrate.

Further provided is a pre-filled syringe comprising a formulation as disclosed herein. The formulations disclosed herein can be used to induce anesthesia or sedation via, e.g., injection or infusion administration to a subject in need of anesthesia or sedation.

### BRIEF DESCRIPTION OF THE FIGURES

Reference is made to the following description of an exemplary embodiment of the present invention, and to the accompanying drawings, wherein:

FIG. 1 shows CPMM solubility in different aqueous buffer media.

FIG. 2 shows log solubility curve of CPMM.

FIG. 3 shows 1- and 2-week stability of CPMM in aqueous pH buffer media stored at 25° C. under 60% relative humidity.

FIG. 4 shows 1- and 2-week stability of CPMM in aqueous pH buffer media stored at 40° C. under 75% relative humidity.

FIG. 5 shows 1- and 2-week stability of CPMM in aqueous pH buffer media stored at 25° C. under 60% relative humidity or at 40° C. under 75% relative humidity (t=0 normalized).

FIG. 6 shows overlay chromatograms of Formulation SBECD-D at 25° C./60% relative humidity.

### DETAILED DESCRIPTION

An anesthetic formulation for clinical and veterinary use can be an aqueous solution that can afford ease of storage and administration and avoid pain to the patient upon, e.g., intravenous administration. However, in an aqueous solution, CPMM is only moderately soluble the metabolically-labile ester is also somewhat unstable, both of which can lead to degradation of the compound upon storage in an aqueous medium. Provided herein, then, are aqueous CPMM formulations that are stable and/or provide a desired level of solubility of the compound, as discussed in detail below.

Formulations suitable for convenient intravenous injection are also described herein, e.g. ready-to-use formulations or lyophilized formulations that can be reconstituted for administration. Further provided are injectable formulations of these compounds, e.g., CPMM at adequate concentration and/or stability that can be suitable for intravenous injection in humans and animals to induce and maintain monitored anesthesia care (MAC) and/or general anesthesia in patients or animals undergoing diagnostic or therapeutic procedures and/or sedation.

The compositions, formulations, and methods are contemplated to include embodiments covering any combination of one or more of the additional optional elements, features, and steps further described below, unless stated otherwise.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

The lyophilized formulation can be reconstituted with an aqueous diluent prior to administration. Non-limiting examples of diluents include saline, water, and buffer. The lyophilized formulation comprises the salt form of an active agent, a solubilizer, and optionally a buffer, acid, and/or base.

The aqueous formulation can be made to have a pH that is tolerable to a subject upon administration, e.g., by injection or infusion. For example, the pH can be in a range of about 2 to about 7. In the case of a lyophilized formulation, the pH is a property of the formulation after reconstitution.

CPMM, which has an IUPAC chemical name of 1-(methoxycarbonyl)cyclopropyl-1-[(1R)-1-phenylethyl]-1H-imidazole-5-carboxylate, has the following structure:

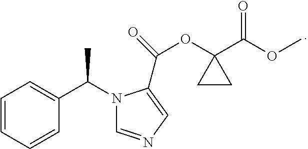 [SMILES: COC(=O)C1(OC(=O)c2cncn2[C@H](C)c2ccccc2)CC1]

CPMM, or a pharmaceutically acceptable salt thereof, is the active compound in the formulations disclosed herein.

As used herein, the term “pharmaceutically-acceptable salts” refers to the conventional non-toxic salts or quaternary ammonium salts of compounds described herein, e.g., from non-toxic organic or inorganic acids. These salts can be prepared in situ in the administration vehicle or the dosage form manufacturing process, or by separately reacting a compound described herein in its free base or acid form with a suitable organic or inorganic acid or base, and isolating the salt thus formed during subsequent purification. Conventional non-toxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. See, for example, Berge et al., “Pharmaceutical Salts”, J. Pharm. Sci. 66:1-19 (1977), the content of which is herein incorporated by reference in its entirety. Exemplary salts also include the hydrobromide, hydrochloride, sulfate, bisulfate, phosphate, nitrate, acetate, succinate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and the like. In certain embodiments, the pharmaceutically acceptable salt can be the hydrochloride salt.

Suitable acids that are capable of forming salts with CPMM include inorganic acids such as hydrochloric acid, hydrobromic acid, perchloric acid, nitric acid, thiocyanic acid, sulfuric acid, phosphoric acid, and the like; and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid, trifluoroacetic acid, methansulfonic acid, benzenesulfonic acid, p-toulenesulfonic acid, and the like.

The compound can be present in a liquid formulation as disclosed herein at a concentration of about 0.1 mg/mL to about 20 mg/mL, or about 1 mg/mL to 10 mg/mL. In some cases, the concentration is about 2 mg/mL to about 5 mg/mL. Since the compound can be administered as a short-acting anesthetic and continuously administered while anesthesia is desired, a subject is often administered high amounts of the compound. Thus, high concentrations of the compound in a formulation are contemplated to minimize the total volume of formulation administered and minimize the total amounts of other excipients (e.g., solubilizers) administered. In the case of lyophilized formulations, the concentration refers to the amount of compound once reconstituted, or as instructed for reconstitution, e.g., by associated labeling. The concentration of the compound can be about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL.

### Solubility, pH, and Solubilizers

The solubility of compounds as disclosed herein is pH-dependent. The imidazole nitrogen in position 3 of CPMM has a pKa of approximately 2.5, as determined by solubility analysis and titration. For example, CPMM is very water soluble at low pH, but solubility decreases with increasing pH, such that it is lower at pH 3-8. Thus, the compound as disclosed herein at lower pHs (e.g., 4 or less, or about 2.5 to about 4) has a higher solubility in aqueous liquid formulations.

The solubilizer for use in the formulation will be pharmaceutically acceptable, e.g., for injection. The solubilizer can be a cyclodextrin. The solubilizer can additionally or alternatively be a co-solvent. Contemplated co-solvents include one or more of ethanol, t-butyl alcohol, DMSO, glycerol, propylene glycol, and polyethylene glycol.

The compositions disclosed herein can also contain at least one cyclodextrin. Cyclodextrin is a cyclic structure of sugar units, typically having 6 (α-cyclodextrin), 7 (β-cyclodextrin), 8 (γ-cyclodextrin), or 9 (δ-cyclodextrin) sugar units in one cyclodextrin molecule. Also contemplated are cyclodextrins having 5, 10, 11, 12, 13, or more sugar units.

Cyclodextrins may be amorphous or crystalline. Cyclodextrins are commercially available, or may be synthesized via means well known in the art. Examples of useful cyclodextrins include, but are not limited to, modified or unmodified α-, (β-, γ-, and δ-cyclodextrins. Derivatives of cyclodextrins include derivatives wherein some of the OH groups are converted to OR groups. Cyclodextrin derivatives include those with short chain alkyl groups such as methylated, ethylated, propylated, and butylated cyclodextrins, wherein R is a methyl, ethyl, propyl, or butyl group; those with hydroxyalkyl substituted groups, such as, e.g., hydroxypropyl cyclodextrins and/or hydroxyethyl cyclodextrins, wherein R is a —CH₂CH(OH)CH₃ or a —CH₂CH₂OH group; branched cyclodextrins such as maltose-bonded cyclodextrins; cationic cyclodextrins such as those containing 2-hydroxy-3-(dimethylamino)propyl ether, wherein R is CH₂CH(OH)CH₂N(CH₃)₂; quaternary ammonium, e.g., 2-hydroxy-3-(trimethylammonio)propyl ether chloride groups, wherein R is —CH₂CH(OH)CH₂N+(CH₃)₃Cl—; anionic cyclodextrins such as carboxymethyl cyclodextrins, cyclodextrin sulfates, and cyclodextrin succinylates; amphoteric cyclodextrins such as carboxymethyl/quaternary ammonium cyclodextrins; cyclodextrins wherein at least one glucopyranose unit has a 3-6-anhydro-cyclomalto structure, e.g., mono-3-6-anhydrocyclodextrins, as disclosed in “Optimal Performances with Minimal Chemical Modification of Cyclodextrins”, F. Diedaini-Pilard et al., The 7th International Cyclodextrin Symposium Abstracts, April 1994, p 49; and mixtures thereof. Other specific modifications contemplated include one or more hydroxyalkyl ether (e.g., R is C₁₋₆alkylenehydroxy) moieties; one or more sulfoalkyl ether (e.g., R is C₂₋₆alkyleneSO₃—) moieties; carboxyalkyl (e.g., R is C(O)C₁₋₆alkyl) moieties; 6-perdeoxy-6-per(4-carboxyphenyl)thio moieties (Cooper et al., Org. Biomol. Chem., 3:1863 (2005)); substituted phenoxy moieties, such as 6-O-phenyl, 6-O(4-formyl-phenyl), 6-O-(4-nitrophenyl), 6-O-(4-bromophenyl), 6-O-(4-chlorophenyl), and 6-O-(4-hydroxybenzyl) (Liu et al., J. Org. Chem., 69:173 (2004)); 6-amino-6-deoxy cyclodextrins (Rekharsky et al., J. Am. Chem. Soc., 124:12361 (2002)); tryptophan moieties (Wang et al., J. Org. Chem. 70:8703 (2005)); or mixtures thereof.

Cyclodextrin derivatives suitable for use herein include hydroxypropyl α-cyclodextrin, methylated α-cyclodextrin, methylated β-cyclodextrin, hydroxyethyl β-cyclodextrin, and hydroxypropyl β-cyclodextrin. A known methylated β-cyclodextrin is heptakis-2,6-di-O-methyl-β-cyclodextrin, commonly known as DIMEB, in which each glucose unit has about 2 methyl groups with a degree of substitution of about 14. Another commercially available cyclodextrin which can be used in the disclosed compositions is methylated β-cyclodextrin, a randomly methylated β-cyclodextrin, commonly known as RAMEB.

Other modified cyclodextrins are described, for example, in U.S. Pat. Nos. 3,426,011; 3,453,257; 3,453,258; 3,453,259; 3,453,260; 3,459,731; 3,453,257; 3,420,788; 3,426,011; 3,553,191; 3,565,887; 4,535,152; 4,616,008; 4,638,058; 4,678,598; 4,727,064; 4,746,734; 5,376,645; 5,134,127; 5,376,645; 5,602,112; 5,804,568; 5,824,668; 5,874,418; 6,046,177; 6,048,845; 6,133,248; 6,136,846; 6,218,374; 6,284,747; 6,509,370; 6,583,125; and 6,982,253, each incorporated by reference in its entirety herein.

In the formulations described herein, the cyclodextrins can be α-cyclodextrins, β-cyclodextrins, γ-cyclodextrins, and/or δ-cyclodextrins. The cyclodextrins can be modified cyclodextrins (e.g. a modified α-cyclodextrin, β-cyclodextrin, γ-cyclodextrin, and/or β-cyclodextrin). Specific modifications include, but are not limited to, hydroxyalkyl ethers and sulfoalkyl ethers. The cyclodextrins can be one or more of hydroxypropyl-β-cyclodextrin (HPBCD or HPβCD interchangeably) and sulfobutylether-β-cyclodextrin (SBEBCD or SBEβCD interchangeably). The modified cyclodextrins can be sulfobutylether-1-β-cyclodextrin, sulfobutylether-4-β-cyclodextrin, sulfobutylether-7-β-cyclodextrin, and/or hydroxypropylether-β-cyclodextrin. The modified cyclodextrin can comprise sulfobutylether-7-β-cyclodextrin, for example. In some cases, the cyclodextrin is sulfobutylether-β-cyclodextrin, hydroxypropyl-β-cyclodextrin, or a combination thereof.

The amount of solubilizer, e.g., cyclodextrin, in a composition disclosed herein may be widely adjusted to achieve desired physical characteristics, such as solubility, stability, and/or decreased toxicity of the formulation. The solubilizer, e.g., cyclodextrin, can be present in an amount in a range of about 6% to about 30%, about 6% to about 20%, or about 6% to about 12% by weight based on weight of cyclodextrin per volume (% w/v) or weight (% w/w) of solution. Specific weight percentages of cyclodextrin present in a composition of the present invention can include about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30% weight by weight (% w/w) or weight by volume (% w/v), for example.

The solubilizer can be present in a molar ratio to the compound of about 1 mol solubilizer to 1 mol compound (CPMM) (1:1) to about 8 mol solubilizer to 1 mol compound (8:1). In some cases, the ratio or solubilizer to compound is about 1:1 to about 4:1. In cases where the solubilizer is a cyclodextrin, the ratio of compound complexed to the cyclodextrin to uncomplexed compound is 1.5 mol complexed to 1 mol uncomplexed to 16 mol complexed to 1 mol complexed (1.5:1 to 16:1). In some cases, the ratio of complexed to uncomplexed is 2:1 to 15:1, 3:1 to 15:1, 10:1 to 15:1, or 5:1 to 16:1.

The pH of an aqueous solution of a salt of a compound as disclosed herein, e.g., CPMM, is in a range of about 2.5 to about 4, based upon the pKa of the CPMM salt. The solubilizer can allow for the maintenance of or increased solubility and/or stability of the compound in a liquid formulation at pH greater than 2.5, about 2.5 to about 4, about 3 to about 7, or about 6 to about 7.4. The pH of the aqueous formulation can be adjusted by inclusion of a buffer and/or pH adjuster (e.g., an acid or base). The pH of the aqueous formulation can be in a range of about 2 to about 7, about 2.5 to about 7.4, about 2.5 to about 4, about 4 to about 6.5, about 5 to about 6.5, or about 6 to about 7.4.

The buffer or base can be an amine-based buffer or base. The buffer will be one that is pharmaceutically acceptable, e.g., for an injectable formulation. Specific buffers, acids, and bases contemplated include, but are not limited to, NaOH, KOH, triethylamine, meglumine, diethanolamine, ammonium hydroxide, ammonium acetate, arginine, lysine, histidine, a phosphate buffer (e.g., sodium phosphate tribasic, sodium phosphate dibasic, sodium phosphate monobasic, or o-phosphoric acid), sodium bicarbonate, a Britton-Robinson buffer, a Tris buffer (containing Tris(hydroxymethyl)aminomethane), a HEPES buffer (containing N-(2-hydroxyethyl)piperazine-N′-(2-ethanesulfonic acid), acetate, a citrate buffer (e.g., citric acid, citric acid anhydrous, citrate monobasic, citrate dibasic, citrate tribasic, citrate salt), ascorbate, glycine, glutamate, lactate, malate, formate, sulfate, and mixtures thereof. In some cases, the buffer comprises a citrate buffer and NaOH. Buffer salts can be anhydrous or hydrates. Buffers can be sodium salts or potassium salts.

### Stability

The aqueous formulations disclosed herein can be storage stable, as assessed by the total amount of degradants measured and the amount of any single degradant after a period of time following making the formulation from the pure active compound. The formulation can exhibit 5% or less, 4% or less, 3% or less, 2% or less, or 1% or less total degradants after a particular time period under particular temperatures, and optionally elevated humidity. The degradant level(s) can be assessed as an absolute measurement or as a relative measurement, based upon the starting purity of the active compound, e.g., an increase in degradants of 5% or less, 4% or less, 3% or less, 2% or less, or 1% or less, relative to the starting amounts in the original active compound. The formulation is assayed and the amount of degradants and active agent are measured to determine the stability of the formulation under the specific storage conditions. The stability can be assessed after 1 hour, 4 hours, 6 hours, 8 hours, 12 hours, 18 hours, 24 hours, 2 days, 3 days, 1 week, 2 weeks, 1 month, 2 months, 3 months, 4 months, 5 months, 6 months, 7 months, 8 months, 9 months, 10 months, 11 months, 1 year (12 months), 18 months, 2 years (24 months), 2.5 years (30 months), or 3 years (36 months) or longer. The temperature at which the formulation is held to assess stability can be at less than 0° C., less than −10° C., in a range of about −20° C. to −10° C., in a range of about 2° C. to about 8° C., or about 15° C. to about 30° C. (e.g., room temperature). In cases where the formulation is lyophilized, the formulation is reconstituted with an aqueous diluent just prior to assessing the amount of degradants. The stability of the formulation can be assessed after 1 week stored at less than −10° C. or −20° C. to −10° C.; after 1 month stored at less than −10° C. or −20° C. to −10° C.; after 3 months stored at less than −10° C. or −20° C. to −10° C.; after 6 months stored at less than −10° C. or −20° C. to −10° C.; after 1 year stored at less than −10° C. or −20° C. to −10° C.; after 1 week stored at less than 0° C.; after 1 month stored at less than 0° C.; after 3 months stored at less than 0° C.; after 6 months stored at less than 0° C.; after 1 year stored at less than 0° C.; after 1 week stored at 2-8° C.; after 1 month stored at 2-8° C.; after 3 months stored at 2-8° C.; after 6 months stored at less than 2-8° C.; after 1 year stored at 2-8° C.; after 1 week stored at 16-28° C.; after 1 month stored at 15-30° C.; after 3 months stored at 15-30° C.; after 6 months stored at 15-30° C.; after 1 year stored at 15-30° C.; or a combination thereof.

### Methods of Making Formulations

Cyclodextrin-based formulations can be obtained by preparing a solution of a cyclodextrin (8-20% w/w), to which is added the active compound (0.1-20 mg/mL). The pH can then be adjusted to the desired level (pH 2.5-7.4) using an appropriate acid, base, or buffer. The resulting solution can be filtered to sterility into a container. Alternatively, cyclodextrin-based formulations can be obtained by preparing a cyclodextrin solution having the desired level of pH before adding the active compound. The solution may be used immediately or stored under appropriate conditions, e.g., frozen (approximately −20° C. or −20° C. to −10° C.), refrigerated (2-8° C.) or at ambient temperature (15-30° C.) depending upon the duration of storage.

Optionally, the formulation can be lyophilized after preparation and stored under appropriate conditions, e.g., frozen (approximately −20 to −10° C.), refrigerated (2-8° C.) or at ambient temperature (16-28° C.) depending upon the duration of storage. Prior to use (e.g., 24 hours or less, or 12 hours or less, or 6 hours or less, or 2 hours or less), the healthcare professional can reconstitute the lyophilized formulation, as described above. The lyophilized formulation can further include one or more modifiers. The modifier can be selected from one or more members of each of the following categories, and one or more agent in each category: bulking agents, tonicifying agents, antimicrobial agents, antioxidants, and collapse temperature modifiers. Bulking agents include sugars (mannitol, lactose, sucrose, trehalose, sorbitol, glucose, raffinose), amino acids (arginine, glycine, histidine), and polymers (polyethylene glycol (PEG), dextran, polyvinylpyrrolidone (PVP)). Tonicifying agents include sodium chloride, sucrose, mannitol, and dextrose. Antimicrobial agents include benzyl alcohol, phenol, m-cresol, methyl paraben, and ethyl paraben. Antioxidants include ascorbic acid, glutamate, sulfite, and bisulfite. Collapse temperature modifiers include dextran, hydroxyethyl starch, ficoll, and gelatin. The modifier can be added to the formulation prior to lyophilization. Non-limiting examples of modifiers for use in lyophilized formulations include dextran, hydroxyethyl starch, hydroxymethyl starch, Ficoll, gelatin, mannitol, lactose, sucrose, trehalose, sorbitol, glucose and raffinose.

A formulation as disclosed herein can be placed into a syringe, as a “pre-filled syringe,” for later use.

### Methods of Administration

For intravenous administration, a formulation as disclosed herein can be drawn into a syringe or filled in an intravenous infusion bottle or bag and administered to the human or animal as a bolus and/or continuous infusion. To induce brief periods of anesthesia or induce anesthesia to be maintained for longer durations, bolus administration can be made manually with a formulation from a syringe. To achieve extended duration of anesthesia, continuous infusion administration can be made either from a syringe mounted in a syringe pump set to deliver formulation at an appropriate rate, or from an intravenous bag or bottle with administration rate regulated by a drip rate or peristaltic pump. Typically, bolus administration can be used to induce anesthesia prior to continuous infusion to sustain anesthesia. Bolus administration and infusion administration are each contemplated for inducing sedation.

The formulations are administered so that the active compound is used or given at a dose from 1 μg/kg to 1000 mg/kg; 1 μg/kg to 500 mg/kg; 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg.

It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. It is to be further understood that the ranges intermediate to those given above are also within the scope of this disclosure, for example, in the range 1 mg/kg to 10 mg/kg, for example use or dose ranges such as 2 mg/kg to 8 mg/kg, 3mg/kg to 7 mg/kg, 4 mg/kg to 6 mg/kg, and the like.

The formulation can be administered as a single bolus or multiple boluses, as a continuous infusion, or a combination thereof. For example, the formulation can be administered as a single bolus initially, and then administered as a continuous infusion following the bolus. The rate of the infusion can be any rate sufficient to affect anesthesia or sedation. Some contemplated infusion rates include from 1 μg/kg/min to 100 mg/kg/min, or from 1 μg/kg/hr to 1000 mg/kg/hr. It will be appreciated that the rate of infusion can be determined based upon the dose necessary to induce sedation or anesthesia and the rate of elimination of the compound, such that the formulation is administered via infusion at a rate sufficient to safely maintain a sufficient amount of compound in the bloodstream to affect anesthesia or sedation.

In some embodiments, the formulation is used or administered at a dosage so that the active compound has an in vivo concentration of less than 500 nM, less than 400 nM, less than 300 nM, less than 250 nM, less than 200 nM, less than 150 nM, less than 100 nM, less than 50 nM, less than 25 nM, less than 20, nM, less than 10 nM, less than 5 nM, less than 1 nM, less than 0.5 nM, less than 0.1 nM, less than 0.05 nM, less than 0.01 nM, less than 0.005 nM, or less than 0.001 nM, at and/or after a specific time following use or administration, such as 15 mins, 30 mins, 1 hr, 1.5 hrs, 2 hrs, 2.5 hrs, 3 hrs, 4 hrs, 5 hrs, 6 hrs, 7 hrs, 8 hrs, 9 hrs, 10 hrs, 11 hrs, 12 hrs or more of time after use or administration of the composition. For example, the active compound in a formulation as disclosed herein is administered at a dosage so that it has an in vivo concentration of less than 500 nM at 30 minutes after use or administration. As another example, the active compound is administered at a dosage so that its inactive metabolite has an in vivo concentration of less than 500 nM at 1 hr after use or administration.

In some cases, the formulation is used or administered at a dosage so that the active compound has an initial in vivo concentration of about 1 μM to about 10 μM to achieve anesthesia, then is administered as an infusion to maintain anesthesia. After administrations of the infusion, the compound concentration drops to less than 500 nM as noted above.

The terms “administration of” and or “administering” a compound should be understood to mean providing a compound or a composition described herein to a subject in need of inducing anesthesia. As such, the term “administer” refers to the placement of a compound or composition described herein into a subject by a method or route which results in at least partial localization of the compound or composition at a desired site such that general anesthesia or conscious sedation is induced and/or maintained in the subject.

### EXAMPLES

### Example 1

Early work showed that CPMM quickly hydrolyzed into etomidate acid in an aqueous solution. Studies were performed to develop a CPMM formulation that slowed this hydrolysis reaction.

### Aqueous pH Solubility

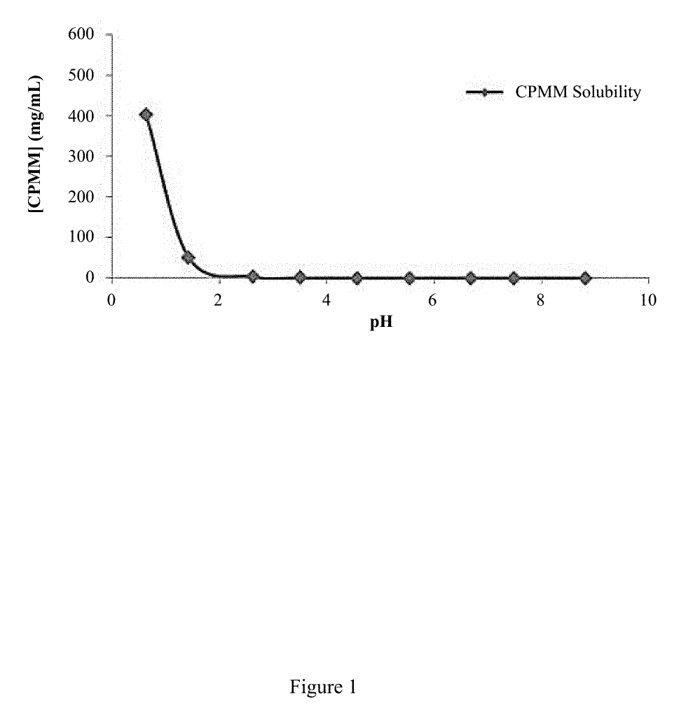

CPMM was evaluated for maximum solubility in aqueous pH 1-9 buffer solutions. CPMM was weighted into 1.0 mL of each solution, vortex mixed to solubilize, and placed on a shaker table until saturated. Additional CPMM was added and replaced on the shaker table until a saturated solution was obtained. The one exception was the pH 1 sample solution, which never reached saturation level due to the CPMM's high solubility at that pH range. Saturated solutions were centrifuged and the supernatants were analyzed by HPLC. The maximum CPMM concentrations of each aqueous buffer solution is listed in Table 1 and plotted in FIG. 1.

TABLE 1
Maximum solubility of CPMM in aqueous pH 1-9 buffer media.

| | | Actual [CPMM] | |
|---|---|---|---|
| | | Concentration | |
| 50 mM Buffered Sample | Actual pH | (mg/mL) | Log Solubility |
| pH 1 (phosphoric acid) | 0.63 | 403.535* | 2.605881 |
| pH 2 (phosphoric acid) | 1.43 | 49.362 | 1.698640 |
| pH 3 (phosphoric acid) | 2.62 | 3.148 | 0.498035 |
| pH 4 (citric acid) | 3.5 | 0.677 | −0.169411 |
| pH 5 (citric acid) | 4.57 | 0.325 | −0.488117 |
| pH 6 (citric acid) | 5.55 | 0.284 | −0.546682 |
| pH 7 (phosphoric acid) | 6.68 | 0.268 | −0.571865 |
| pH 8 (phosphoric acid) | 7.48 | 0.260 | −0.585027 |
| pH 9 (phosphoric acid) | 8.81 | 0.279 | −0.554396 |

*not a true maximum solubility concentration

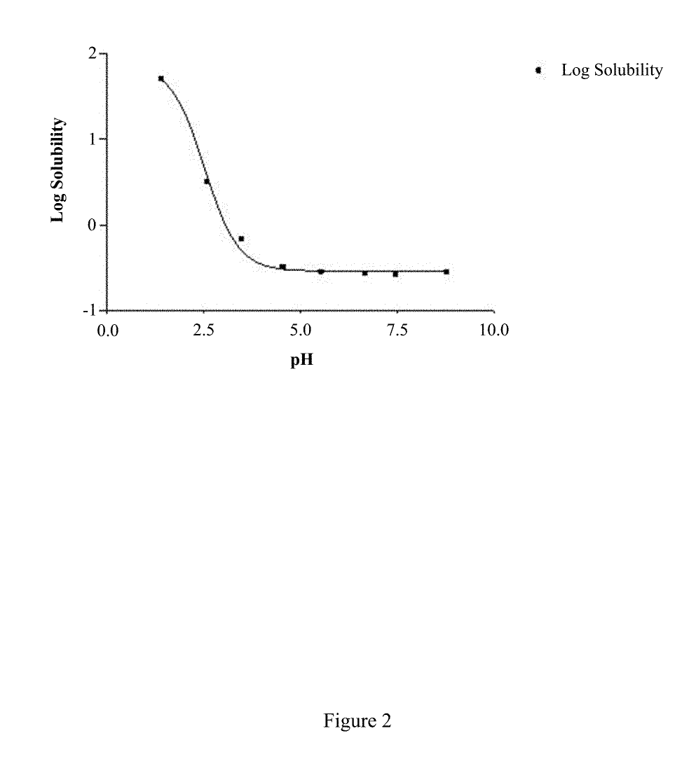

The CPMM solubility data was used to generate a log solubility curve, which is shown in FIG. 2. Based on this data, the pKa was estimated to be about 2.5.

CPMM solubility was also evaluated in common solvents: water, methanol, ethanol, acetonitrile, and isopropanol. CPMM was weighed into 1.0 mL of each solvent and vortex-mixed. Only the water sample was saturated with CPMM; the other solvents never achieved saturation due to their high CPMM solubility. The screening was stopped after 100 mg of bulk CPMM had been added. Table 2 lists the ABP-700 solubility results in common solvents.

TABLE 2
CPMM solubility in common solvents.

| | | | Actual [CPMM] |
|---|---|---|---|
| | | HPLC [CPMM] | Concentration |
| Solvent | Dilution Factor | Concentration (μg/mL) | (μg/mL) |
| Water | 6.67 | 449.2 | 2996.2 |
| Acetonitrile | 166.67 | 474.2 | 79034.9 |
| Methanol | 166.67 | 503.2 | 83868.3 |
| Ethanol | 166.67 | 534.3 | 89051.8 |
| Isopropanol | 166.67 | 514.5 | 85751.7 |

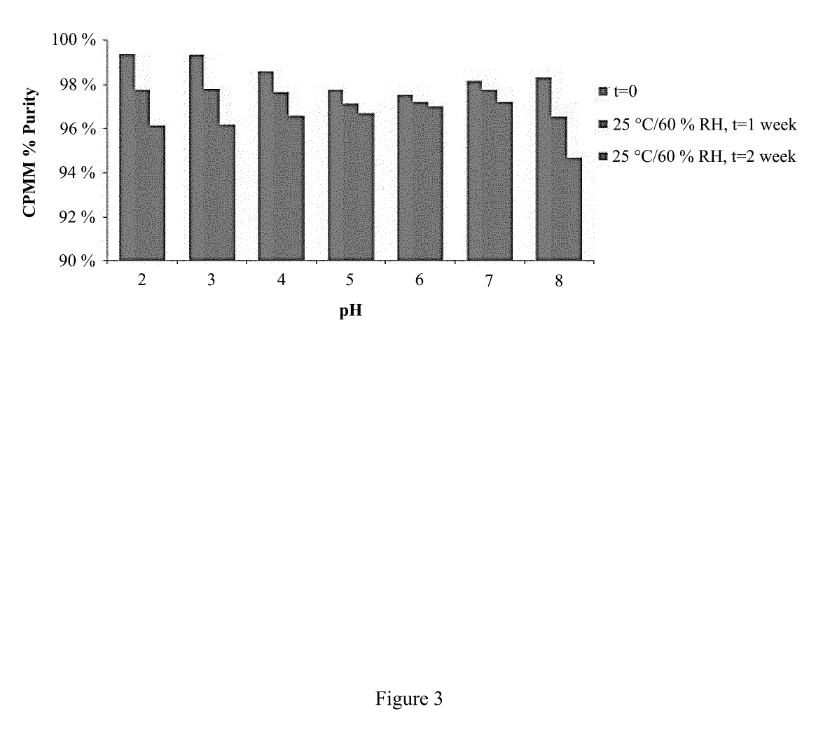

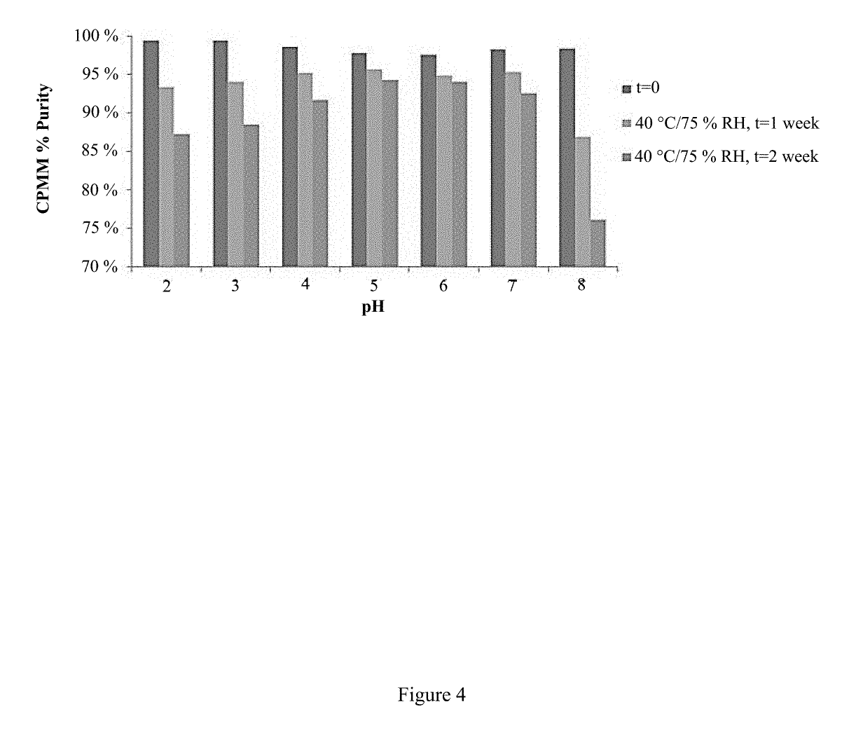

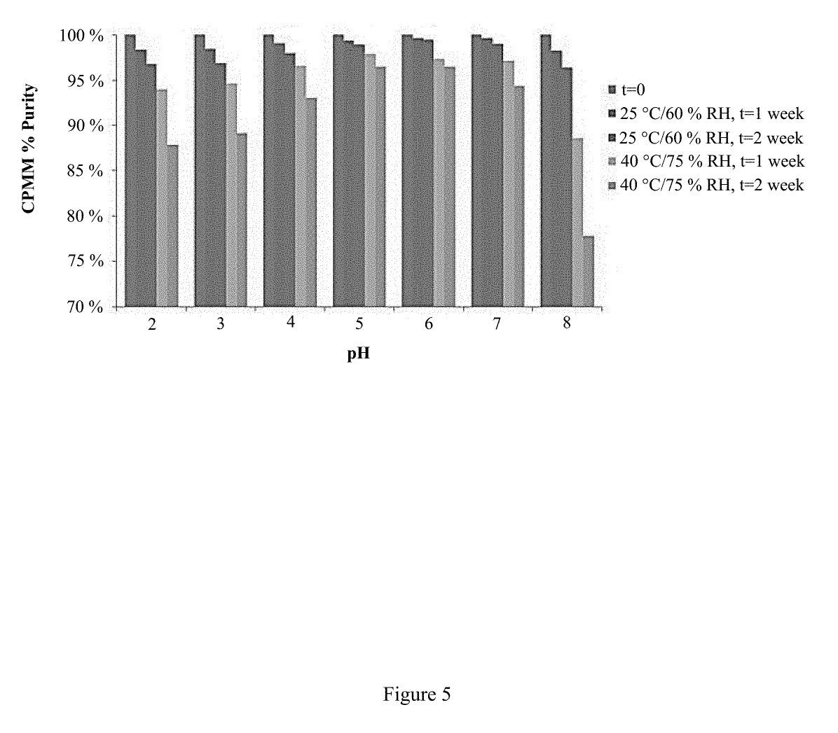

The aqueous CPMM pH 1-9 samples described above were diluted as necessary to approximately 0.3 mg/mL and placed on accelerated stability at 25° C./60% relative humidity and 40° C./75% relative humidity conditions for two weeks. Samples were pulled at t=0, 1, and 2 weeks from set-up, and analyzed by HPLC. Samples were diluted to approximately 0.3 mg/mL, which well below the HPLC method nominal concentration, due to concerns of precipitation during the two week stability study. The injection volume was increased to compensate for the lower drug concentration in solution. The change in CPMM purity over the study duration is listed in Table 3 and plotted in FIGS. 3-5.

The pH 1 sample was placed on stability at a non-diluted concentration due to an error. This sample was re-diluted at the one-week time point to match the concentration in the pH 2-9 samples and re-placed on stability. The pH 9 sample results were not included in FIGS. 3-5 due to excessive degradation and for easier visual assessment over the more relevant pH 2-8 range.

TABLE 3
1- and 2-week stability data for CPMM in aqueous pH buffer media.

| | Change in CPMM % Purity | | | | |
|---|---|---|---|---|---|
| | | 25° C./60% RH | 25° C./60% RH | 40° C./75% RH | 40° C./75% RH |
| 50 mM Buffer Sample | t = 0 | t = 1 week | t = 2 week | t = 1 week | t = 2 week |
| pH 2 (Phosphoric Acid) | 99.39% | −1.64% | −3.25% | −6.03% | −12.13% |
| pH 3 (Phosphoric Acid) | 99.33% | −1.53% | −3.16% | −5.36% | −10.87% |
| pH 4 (Citric Acid) | 98.59% | −0.95% | −2.00% | −3.43% | −6.99% |
| pH 5 (Citric Acid) | 97.77% | −0.62% | −1.06% | −2.16% | −3.48% |
| pH 6 (Citric Acid) | 97.55% | −0.33% | −0.55% | −2.72% | −3.49% |
| pH 7 (Phosphoric Acid) | 98.16% | −0.39% | −0.96% | −2.90% | −5.62% |
| pH 8 (Phosphoric Acid) | 98.32% | −1.76% | −3.62% | −11.39% | −22.20% |
| pH 9 (Boric Acid) | 94.98% | −25.38% | −44.67% | −78.47% | −92.14% |

### Binary Excipient Screening

CPMM was evaluated for compatibility with excipients that are suitable for IV administration. A buffer solution (phosphate at pH 3) was selected as the diluent for excipient screening studies. Table 4 shows the IV-compatible excipients and their respective amounts in the phosphate pH 3 buffer diluent. CPMM was solubilized in each excipient solution at 1 mg/mL.

TABLE 4
IV-compatible excipients and their respective amounts in the phosphate pH 3
buffer diluent.

| | | Amount (% w/w) in 10 mM | |
|---|---|---|---|
| Excipient Type | Excipient Name | pH 3 Phosphate Buffer | [ABP-700]_{Free Base} |
| Co-Solvents | Ethanol | 10% | 1 mg/mL |
| | Glycerol | 10% | 1 mg/mL |
| | PEG 400 | 10% | 1 mg/mL |
| | Propylene Glycol | 10% | 1 mg/mL |
| Surfactants | Polysorbate 20 | 1% | 1 mg/mL |
| | Polysorbate 80 | 1% | 1 mg/mL |
| | Lutrol F68 | 1% | 1 mg/mL |
| | Solutol HS15 | 1% | 1 mg/mL |
| Complexation Agents | HP-β-CD | 10% | 1 mg/mL |
| | SBE-β-CD | 10% | 1 mg/mL |
| Antioxidants/Chelating | Ascorbic Acid | 0.5% | 1 mg/mL |
| Agents | Sodium Bisulfite | 0.5% | 1 mg/mL |
| | Ascorbic Acid/Edetate | 0.5%/0.05% | 1 mg/mL |
| | Disodium Dihydrate | | |
| Bulking Agents, | Mannitol | 5% | 1 mg/mL |
| Stabilizers, Tonicity | Sorbitol | 5% | 1 mg/mL |
| Adjusters | Sucrose | 5% | 1 mg/mL |
| | Trehalose | 5% | 1 mg/mL |
| | Dextrose | 5% | 1 mg/mL |
| | NaCl | 5% | 1 mg/mL |
| | KCl | 5% | 1 mg/mL |
| | PVP K17 | 5% | 1 mg/mL |

At a 1 mg/mL concentration, CPMM readily dissolved in the two cyclodextrin solutions, as well as all of the surfactant solutions. However, slow CPMM solubilization was observed for all other excipient samples during solution preparation. While CPMM completely dissolved in the control sample within several minutes, many of the excipient solutions required overnight storage at 5° C. to solubilize the drug.

Each excipient sample solution was placed on a two-week stability study at 25° C./60% relative humidity and 40° C./75% relative humidity, and analyzed for CPMM purity at t=0, 1, and 2 weeks by comparing individual samples to their respective excipient blank solution. The excipient compatibility data are listed in Table 5.

TABLE 5
1- and 2-week stability data for CPMM in aqueous solution containing
different IV-compatible excipients.

| | | Change in CPMM % Purity | | | | |
|---|---|---|---|---|---|---|
| Excipient | | | t = 1 week | t = 2 weeks | t = 1 week | t = 2 weeks |
| Type | Excipient Name | t = 0 | 25° C./60% RH | 25° C./60% RH | 40° C./75% RH | 40° C./75% RH |
| Control | pH 3 Buffer | 99.5% | −1.6% | −2.6% | −5.3% | −9.2% |
| | Water | 99.4% | −0.9% | Not Determined | −3.4% | Not Determined |
| Co-Solvents | Ethanol | 99.5% | −1.1% | −3.1% | −3.2% | −7.0% |
| | Glycerol | 99.4% | −1.3% | −3.6% | −4.7% | −9.8% |
| | PEG 400 | 99.5% | −1.2% | −2.0% | −4.0% | −7.0% |
| | Propylene Glycol | 99.4% | −1.2% | −1.9% | −4.0% | −6.9% |
| Surfactants | Polysorbate 20 | 99.4% | −1.4% | −2.1% | −4.6% | −8.1% |
| | Polysorbate 80 | 99.4% | −4.2%^{†} | −5.2%^{†} | −8.3%^{†} | −15.0%^{†} |
| | Lutrol F68 | 99.5% | −1.6% | −2.5% | −5.2% | −9.0% |
| | Solutol HS15 | 99.4% | −1.3% | −2.1% | −4.2% | −7.3% |
| Complexation | HPβCD | 99.4% | −0.4% | −0.6% | −1.6% | −2.5% |
| Agents | SBECD | 99.4% | −0.4% | −0.4% | −1.3% | −2.1% |
| Antioxidants/ | Ascorbic Acid | 97.6% | −14.4% | −26.4% | −31.6% | −49.8% |
| Chelating | Sodium Bisulfite | 99.4% | −1.9% | −3.2% | −7.2% | −12.2% |
| Agents | Ascorbic Acid/ | 99.4% | −2.2% | −4.2% | −9.8% | −17.8% |
| | Edetate Disodium | | | | | |
| | Dihydrate | | | | | |
| Bulking | Mannitol | 99.4% | −1.5% | −2.6% | −5.2% | −9.1% |
| Agents, | Sorbitol | 99.5% | −1.3% | −2.3% | −4.5% | −8.3% |
| Stabilizers, | Sucrose | 99.4% | −1.5% | −2.5% | −5.2% | −9.0% |
| Tonicity | Trehalose | 99.4% | −1.3% | −2.3% | −4.6% | −8.4% |
| Adjusters | Dextrose | 99.4% | −1.5% | −2.5% | −5.2% | −9.0% |
| | NaCl | 99.4% | −1.1% | −1.9% | −4.0% | −7.2% |
| | KCl | 99.4% | −1.3% | −2.2% | −4.8% | −8.2% |
| | PVP K17 | 99.6%* | ND* | ND* | ND* | ND* |

*A large excipient related peak elutes at a similar retention time several rel. substances and are not accounted for in these purity values.
^{†}Increased levels of degradation may be due to excipient impurities.
RH: Relative Humidity

### ND: Not Detected

Complexation agents at pH 3 were the only excipients that were found to be more stabilizing than the water control sample at neutral pH. All other excipient solutions either only minimally stabilized CPMM or de-stabilized CPMM also at pH 3.

### Example 2

Based on the excipient screening and pH solubility data, initial studies focused on the use of cyclodextrins to solubilize CPMM in an aqueous formulation. Selected test formulations containing are HPβCD and SBECD are described in Tables 6 and 7, respectively.

TABLE 6
Summary of results from initial studies on select test HPβCD formulations.

| | | Target | | | Initially | |
|---|---|---|---|---|---|---|
| Form | Formulation | [CPMM], | | | Soluble at | Extended |
| No. | Composition | (mg/mL)* | pH | Observations | pH 3.0? | Solubility |
| 1 | 10% HPβCD | 30 | Initial | Slowly adjusted pH with 1N | No | Not |
| | in water | | pH: 1.5 | NaOH. | | Determined |
| | | | Final | At pH 1.76: solution remained | | |
| | | | pH: 1.9 | clear, no precipitation. | | |
| | | | | At pH 1.87: immediately cloudy | | |
| | | | | precipitation observed. | | |
| 2 | 20% HPβCD | 20 | pH: 1.9 | CPMM was completely soluble. | Not | Not |
| | in 10 mM | | | No pH adjustment was made. | Determined | Determined |
| | PBS, pH 7.4 | | | | | |
| 3 | 20% HPβCD | 20 | pH: 2.4 | CPMM was completely soluble. | Not | Not |
| | in 100 mM | | | No pH adjustment was made. | Determined | Determined |
| | tartrate buffer, | | | | | |
| | pH 3 | | | | | |
| 4 | 20% HPβCD | 8 | Initial | Pre-adjusted formulation was a | Yes | Not |
| | in water | | pH: 2.1 | clear solution. | | Determined |
| | | | Final | Adjusted pH using 1N NaOH to | | |
| | | | pH: 7.8 | pH 7.8. | | |
| | | | | Final solution remained clear. | | |
| 5 | 20% HPβCD | 20 | Initial | Slowly adjusted pH with 0.1N | Yes | Not |
| | in water | | pH: 1.8 | NaOH. | | Determined |
| | | | Final | At pH 3.28: solution remained | | |
| | | | pH: 3.3 | clear, no precipitation. | | |
| | | | | At pH 3.32: immediately formed | | |
| | | | | white precipitate. | | |
| | | | | Large pH fluctuations observed | | |
| | | | | above ~pH 3.5 since solution is | | |
| | | | | non-buffered at that range. | | |
| 6 | 20% HPβCD | 20 | Initial | Slowly adjusted pH with 0.1N | Yes | Not |
| | in 1% acetic | | pH: 1.8 | NaOH. | | Determined |
| | acid in water | | Final | At pH 3.2: solution remained | | |
| | | | pH: 3.4 | clear with no precipitation. | | |
| | | | | At pH 3.4: immediately saw | | |
| | | | | very small fine particles, which | | |
| | | | | continued to precipitate out more | | |
| | | | | over the course of ~5 min. | | |
| 7 | 20% HPβCD | 20 | Initial | Slowly adjusted pH with 1N | Yes | Not |
| | in 1% acetic | | pH: 1.8 | NaOH. | | Determined |
| | acid in water | | Final pH | At pH 3.1: solution remained | | |
| | | | 3.4 | clear with no precipitation. | | |
| | | | | At pH 3.4: observed insoluble | | |
| | | | | precipitates after the last addition | | |
| | | | | of 1N NaOH. | | |
| 8 | 20% HPβCD | 20 | Initial | Prep 1: adjusted pH with 1N | Yes | Physically |
| | in water | | pH: 1.8 | NaOH. | | stable for ~6 |
| | | | Final | At pH 3.0: solution remained | | hours |
| | | | pH: 3.0 | clear, no precipitation. | | at RT and 3 |
| | | | | Solution is physically stable at | | days when |
| | | | | room temperature for at least ~4-6 | | filtered |
| | | | | hours. Crystalline precipitate | | and stored at |
| | | | | observed after that time. | | 2-8° C. |
| | | | | Prep 2: adjusted pH with 1N | | |
| | | | | NaOH. | | |
| | | | | At pH 3.0: solution remained | | |
| | | | | clear, no precipitation. | | |
| | | | | Filtered solution through a | | |
| | | | | 0.2 μm PVDF filter - and stored | | |
| | | | | in new glass vials at room | | |
| | | | | temperature and 2-8° C. The | | |
| | | | | filtered room temp solution was | | |
| | | | | stored overnight and crystalline | | |
| | | | | precipitates were observed the | | |
| | | | | following morning (~15 hours). | | |
| | | | | The filtered 2-8° C. solution | | |
| | | | | remained completely soluble for | | |
| | | | | 3 days. | | |
| 9 | 20% HPβCD | 25 | Initial | Slowly adjusted pH with 1N | No | Not |
| | in water | | pH: 1.8 | NaOH. | | Determined |
| | | | Final pH | At pH 2.8: solution remained | | |
| | | | 2.9 | clear with no precipitation. | | |
| | | | | At pH 2.9: immediately saw very | | |
| | | | | small fine particles, which | | |
| | | | | continued to precipitate out more | | |
| | | | | over the course of ~5 min. | | |
| 10 | 0.25% HPMC | 25 | Initial | Slowly adjusted pH with 1N | Yes | Not |
| | in 20% | | pH: 1.7 | NaOH. | | Determined |
| | HPβCD in | | Final pH | At pH 3.1: solution remained | | |
| | water | | 3.2 | clear with no precipitation. | | |
| | | | | At pH 3.2: immediately saw | | |
| | | | | small particles, which continued | | |
| | | | | to precipitate out. | | |
| 11 | 0.25% PVP in | 25 | Initial | Slowly adjusted pH with 1N | Yes | Precipitates |
| | 20% HPβCD | | pH: 1.7 | NaOH. | | within |
| | in water | | Final pH | At pH 3.0: solution remained | | ~60 seconds |
| | | | 3.0 | clear initially, but then started to | | at RT |
| | | | | precipitate within <60 seconds. | | |
| 12 | 20% HPβCD | 30 | Initial | Slowly adjusted pH with 0.1N | No | Not |
| | in 1% acetic | | pH: 1.8 | NaOH. | | Determined |
| | acid in water | | Final pH | At pH 2.8: solution remained | | |
| | | | 2.8 | clear with no precipitation. | | |
| | | | | Added another 20 μL of 0.1N | | |
| | | | | NaOH (pH 2.8): immediately | | |
| | | | | saw very small fine particles, | | |
| | | | | which continued to precipitate | | |
| | | | | out more over the course of | | |
| | | | | ~5 min. | | |
| 13 | 20% HPβCD | 30 | Initial | Added 1N NaOH to adjust to | Target pH is | Precipitates |
| | in water | | pH: 1.8 | pH 2.5: solution initially | 2.5. | after ~10 min |
| | | | Final pH | remained clear with no | Formulation | at RT |
| | | | 2.5 | precipitation, but then began to | is initially | |
| | | | | precipitate after ~10 min. from | soluble at | |
| | | | | the time of prep. | pH 2.5 | |
| 14 | 10% HPβCD | 11.5 | Initial | Added 1N NaOH to adjust the | Yes | Not |
| | in water | | pH: 2.0 | pH to 3.1: solution remained | | Determined |
| | | | Final pH | clear with no precipitation. | | |
| | | | 3.2 | Added additional 1N NaOH | | |
| | | | | (pH 3.2): precipitation observed | | |
| | | | | immediately. | | |
| 15 | 5% HPβCD in | 7.3 | Initial | Added 1N NaOH to adjust the | Yes | Precipitates |
| | water | | pH: 2.2 | pH to 3.0: solution remained | | within |
| | | | Final | clear with no precipitation. | | ~2 min after |
| | | | pH: 3.3 | Added additional 1N NaOH | | preparation |
| | | | | (pH 3.4): precipitation observed | | at RT |
| | | | | immediately. | | |
| 16 | 5% HPβCD in | 6.5 | Initial | Added 1N NaOH to adjust the | Yes | Precipitates |
| | water | | pH: 2.3 | pH to 3.0: solution was initially | | within |
| | | | Final | clear with no precipitation, but | | ~10 min |
| | | | pH: 3.0 | then began to precipitate after | | after |
| | | | | ~10 minutes from the time of | | preparation |
| | | | | prep. | | at RT |

*Target concentration corresponds to the free base form and accounts for HPLC purity but not water content and residual solvents.

TABLE 7
Summary of results from initial studies on select test SBECD.

| | | Target | | | Initially | |
|---|---|---|---|---|---|---|
| Form | Formulation | [CPMM], | | | Soluble at | Extended |
| No. | Composition | (mg/mL)* | pH | Observations | pH 3.0? | Solubility |
| 1 | 20% SBECD | 20 | pH: 2.9 | Observed very small film around | No | Not |
| | in 100 mM | | | the sides of glass wall. No pH | | determined |
| | tartrate buffer, | | | adjustment. | | |
| | pH 3 | | | | | |
| 2 | 20% SBECD | 20 | pH 3.4 | Observed small particles stuck to | NA | Not |
| | in 50 mM | | | sides of glass wall, slightly more | | determined |
| | PBS, pH 7.4 | | | than SBECD Form 1. No | | |
| | | | | additional adjustment made. | | |
| 3 | 20% SBECD | 20 | pH: 5.5 | Observed small precipitates, | NA | Not |
| | in 100 mM | | | more than SBECD Form 2. No | | determined |
| | PBS, pH 7.4 | | | adjustment made. | | |
| 4 | 20% SBECD | 20 | Initial | Slowly added 1N NaOH to | Yes | Not |
| | in water | | pH: 2.5 | pH 3.1: solution remained clear, | | determined |
| | | | Final | no precipitation. | | |
| | | | pH: 3.4 | Added additional 1N NaOH | | |
| | | | | (pH 3.4): immediately formed | | |
| | | | | white precipitate. | | |
| | | | | Large pH fluctuations observed | | |
| | | | | above ~pH 3.5 since solution is | | |
| | | | | non-buffered at that range. | | |
| 5 | 20% SBECD | 20 | Initial | Added 0.1N NaOH to adjust to | Yes | Not |
| | in | | pH: 2.5 | pH 3.3: solution remained clear | | determined |
| | 1% acetic acid | | Final | with no precipitation. | | |
| | in water | | pH: 3.4 | Added additional 0.1N NaOH | | |
| | | | | (pH 3.4): immediately saw very | | |
| | | | | small fine particles, which | | |
| | | | | continued to precipitate out more | | |
| | | | | over the course of ~5 min. | | |
| 6 | 20% SBECD | 25 | Initial | Added 1N NaOH to adjust to | No | Not |
| | in water | | pH: 2.4 | pH 2.8: solution remained clear | | determined |
| | | | Final pH | with no precipitation. | | |
| | | | 2.9 | Added additional 1N NaOH | | |
| | | | | (pH 2.9): immediately saw very | | |
| | | | | small fine particles, which | | |
| | | | | continued to precipitate out more | | |
| | | | | over ~5 min. | | |
| 7 | 5% SBECD in | 7.3 | Initial | Added 1N NaOH to adjust to | Yes | Not |
| | water | | pH: 2.8 | pH 3.6: solution remained clear, | | determined |
| | | | Final | no precipitation. | | |
| | | | pH: 3.8 | Added additional 1N NaOH | | |
| | | | | (pH 3.8): immediately saw small | | |
| | | | | fine particles which continued to | | |
| | | | | precipitate out more over ~5 min. | | |

*Target concentration corresponds to the free base form and accounts for HPLC purity but not water content and residual solvents.

### Example 3

Based on the pH evaluations and studies described in Examples 1 and 2, six CPMM formulations containing HPI3CD or SBECD were prepared and tested. These formulations are shown in Table 8.

TABLE 8
CPMM formulations containing HPβCD or SBECD.

| Leading | | | |
|---|---|---|---|
| Test | | | |
| Form- | | | Target |
| ulation | Formulation Composition | pH | [CPMM]_{Free Base} |
| HPβCD-A | 20% hydroxypropyl-β-cyclodextrin, | 3.0 | 20 mg/mL |
| | 0.90% meglumine in water | | |
| HPβCD-B | 10% hydroxypropyl-β-cyclodextrin, | 2.5 | 15 mg/mL |
| | 0.35% meglumine in water | | |
| SBECD-A | 20% sulfobutylether-β-cyclodextrin, | 3.0 | 20 mg/mL |
| | 0.43% meglumine in water | | |
| SBECD-B | 20% sulfobutylether-β-cyclodextrin, | 2.5 | 30 mg/mL |
| | 0.15% meglumine in water | | |
| SBECD-C | 20% sulfobutylether-β-cyclodextrin, | 2.5 | 40 mg/mL |
| | 0.13% meglumine in water | | |
| SBECD-D | 10% sulfobutylether-β-cyclodextrin, | 2.5 | 20 mg/mL |
| | 0.07% meglumine in water | | |

These six formulations were scaled up, filtered through a 0.2 μm PVDF membrane filter (13 mm), and stored at 2-8° C. and ambient temperature/ambient humidity. These formulations were evaluated for physical and chemical stability over two weeks, with time points at t=0, 1 and 2 weeks. The data from this 2-week stability study are reported in Table 9.

TABLE 9
1- and 2-week stability data for CPMM formulations
containing HPβCD or SBECD.

| | | CPMM % Purity | | | | |
|---|---|---|---|---|---|---|
| | | | | | Ambient | |
| | | | 2-8 C. | | Temp/Humidity | |
| Test | | | t = 1 | t = 2 | t = 1 | |
| Formulation | pH | t = 0 | Week | Weeks | Week | t = 2 Weeks |
| HPβCD-A | 2.96 | 98.9% | 98.9% | 98.8% | 98.7% | 98.6% |
| HPβCD-B | 2.47 | 98.9% | 98.9% | 98.6% | 98.6% | 98.4% |
| SBECD-A | 3.08 | 98.9% | 98.9% | 98.8% | 98.7% | 98.7% |
| SBECD-B | 2.53 | 98.9% | 98.9% | 98.8% | 98.6% | 98.6% |
| SBECD-C | 2.42 | 98.9% | 98.8% | 98.8% | 98.7% | 98.5% |
| SBECD-D | 2.56 | 98.9% | 98.8% | 98.8% | 98.6% | 98.4% |

Formulations containing HPβCD versus SBECD at the same active concentrations resulted in similar CPMM purities on stability.

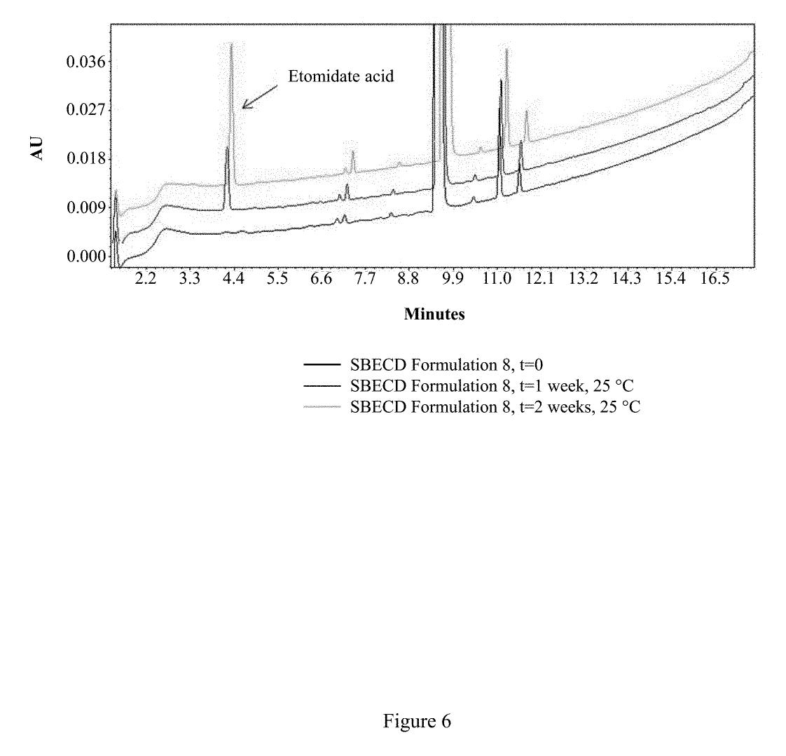

The SBECD formulation remained soluble after multiple freeze-thaw cycles with a consistent pH from t=0. While degradation is observed at or above 25° C./60% relative humidity, the SBECD formulation appeared relatively stable at below ambient temperature conditions. Etomidate acid, which elutes at 4.3 minutes, is the main degradation product for the SBECD formulation at pH 2.5 (see FIG. 6).

### Example 4

A study was conducted to evaluate whether buffer concentration affects the maximum solubility of CPMM. The formulations that were tested contained CPMM, 10% SPECD, and citrate buffer at 10 mM, 25 mM, or 50 mM. The solutions were prepared and CPMM was added until saturation was achieved. The pH was controlled by continually titrating the solutions to pH 6 using NaOH. Saturation was determined to have been achieved after the pH had stabilized and undissolved remained in the vial after overnight shaking. Solubility was evaluated by taking two separate samplings over the course of two days to verify that saturation had been achieved. Solubility was measured when the formulations were at room temperature, as well as tested when the formulations were under sub-ambient conditions (2-8° C.) to evaluate whether the solubility would be affected at lower temperatures for long term storage at (2-8° C.). The results are shown in Table 10.

TABLE 10
Maximum solubility in CPMM formulations with different
concentrations of citrate buffer.

| | | | CPMM Amount |
|---|---|---|---|
| Composition | Condition | pH | (mg/mL) |
| 10 mM Citrate | RT/Ambient RH | 5.96 | 0.30 |
| 25 mM Citrate | RT/Ambient RH | 6.00 | 0.28 |
| 50 mM Citrate | RT/Ambient RH | 5.98 | 0.23 |
| 10% SBECD in | RT/Ambient RH | 5.98 | 5.47 |
| 10 mM Citrate | 2-8° C./ambient RH | 6.05 | 5.48 |
| 10% SBECD in | RT/Ambient RH | 5.98 | 5.49 |
| 25 mM Citrate | 2-8° C./ambient RH | 6.02 | 5.59 |
| 10% SBECD in | RT/Ambient RH | 5.99 | 5.68 |
| 50 mM Citrate | | | |

RT = room temperature
RH = relative humidity

The citrate solutions containing no SBECD had approximately 0.3 mg/mL solubility, which was expected based on previous pre-formulation experiments. The 10% SBECD solutions at all citrate concentrations all had approximately 5.5 mg/mL solubility regardless of the condition. This indicates that the maximum solubility of ABP-700 is most likely not affected by citrate concentration from 10 mM to 50 mM and the storage condition either room temperature or sub-ambient.

### Example 5

Ten CPMM formulations containing SBECD were prepared to study their stability over different storage durations. The components of the formulations are shown in Table 11 below.

TABLE 11
CPMM formulations

| Formu- | | | | | | |
|---|---|---|---|---|---|---|
| lation | % | [CPMM] | | Buffer | | pH |
| # | SBECD | (mg/mL) | pH | Concentration | Scale | Adjuster |
| 1 | 9.0 | 5.0 | 5.0 | 10 mM Citrate | 250 mL | 0.5N |
| 2 | 9.0 | 3.0 | 5.0 | | | NaOH |
| 3 | 11.0 | 5.0 | 6.0 | | | |
| 4 | 11.0 | 3.0 | 5.0 | | | |
| 5 | 9.0 | 5.0 | 6.0 | | | |
| 6 | 10.0 | 4.0 | 5.5 | | | |
| 7 | 9.0 | 3.0 | 6.0 | | | |
| 8 | 10.0 | 4.0 | 5.5 | | | |
| 9 | 11.0 | 3.0 | 6.0 | | | |
| 10 | 11.0 | 5.0 | 5.0 | | | |

The stability of each formulation was assessed, at time points 0.5 months (2 weeks), 1 month, 2 months, 3 months, 6 months, 12 months, 18 months, and 24 months, when stored at (i) 2-8° C. at ambient relative humidity (RH), (ii) 25° C. at 60% RH, and (iii) 40° C. at 75% RH. Each formulation was assessed for potency (e.g., amount of CPMM), pH, related substances (e.g., degradants), and osmolality.

The materials and equipment used to prepare the formulations are shown in Table 12 below.

TABLE 12
Materials and equipment

| Materials and Equipment | Supplier | Comments |
|---|---|---|
| ABP-700 (aka CPMM) | Ricerca Biosciences | Lot No.: 60296-13-001 |
| | | Chloride Content: 10.07% |
| | | Water Content: 1.1% |
| | | Total Impurities: 0.27% |
| In-House Milli-Q Water | Pharmatek | Millipore, Ultrapure, de-ionized |
| Vortex-Genie 2 | Scientific Industries, Inc. | Variable Speed (RPM): 600-3200 |
| Sulfobutylether cyclodextrin (SBECD) | Davos | Manufacturer: Cyclolab |
| | | Lot No.: 47K161212 |
| | | Water Content: 4.1% |
| Sterile Water for Injection (SWFI) | Baxter | NA |
| Sonicator | Bransonic | Model: 5510R-MT |
| | | One Setting for Sonication |
| Citric Acid Anhydrous | EMD¹ | NA |
| Sodium Citrate Tribasic Anhydrous | Spectrum¹ | NA |
| 1.0N NaOH | EMD¹ | NA |

The following steps were used to prepare 10 mM citrate buffer pH 6: 0.00170 mM citric acid, 0.00830 mM sodium citrate (2000 mL scale):
1. Weighed 653.22 mg±13.01 mg of citric acid anhydrous and transferred into a 2000-mL volumetric flask.
2. Weighed 4283.95 mg±20.00 mg of sodium citrate (tribasic) and transferred into the same flask.
3. Diluted to ˜80% volume with SWFI and swirled to dissolve all solids.
4. Adjusted pH with 1N NaOH as necessary to obtain a pH of 6.00±0.05. 5. QS′d to volume and mixed by inversion.

The following steps were used to prepare 10 mM citrate buffer pH 5: 0.00398 mM citric acid, 0.00602 mM sodium citrate, (2000 mL scale):
1. Weighed 1529.31 mg±20.00 mg of citric acid anhydrous and transferred into a 2000-mL volumetric flask.
2. Weighed 3107.15 mg±20.00 mg of sodium citrate (tribasic) and transferred into the same flask.
3. Diluted flask to ˜80% volume with SWFI and swirled to dissolve all solids.
4. Adjusted pH as necessary to obtain a pH of 5.00±0.05. 5. QS′d flask to volume with additional SWFI and mixed by inversion.

The following steps were used to prepare 10 mM citrate buffer pH 5.5: 0.00287 mM citric acid, 0.00713 mM sodium citrate, 1000 mL:
1. Weighed 551.40mg ±11.03 mg of citric acid anhydrous and transferred into a 1000-mL volumetric flask.
2. Weighed 1840.03 mg±20.00 mg of sodium citrate (tribasic) and transferred into the same flask.
3. Diluted flask to ˜80% volume with SWFI and swirled to dissolve all solids.
4. Adjusted pH with IN NaOH as necessary to obtain a pH of 5.50±0.05. 5. QS′d flask to volume with additional SWFI and mixed by inversion.

The following steps were used to prepare 22% SBECD solution in 10 mM citrate, pH 5 (300-g scale):
1. Weighed 68.82 g of SBECD into a 1000-mL solvent bottle.
2. Weighed 231.18 g of 10 mM citrate, pH 5 into the same bottle.
3. Placed on shaker to dissolve all solids.

The following steps were used to prepare 22% SBECD solution in 10 mM citrate, pH 6 (300-g scale):
1. Weighed 68.82 g of SBECD into a 1000-mL solvent bottle.
2. Weighed 231.18 g of 10 mM citrate, pH 6 into the same bottle.
3. Placed on shaker to dissolve all solids.

The following steps were used to prepare 18% SBECD solution in 10 mM citrate, pH 5 (300-g scale):
1. Weighed 56.31 g of SBECD into a 1000-mL solvent bottle.
2. Weighed 243.69 g of 10 mM citrate, pH 5 into the same bottle.
3. Placed on shaker to dissolve all solids.

The following steps were used to prepare 18% SBECD solution in 10 mM citrate, pH 6 (300-g scale):
1. Weighed 56.31 g of SBECD into a 1000-mL solvent bottle.
   2. Weighed 243.69 g of 10 mM citrate, pH 6 into the same bottle.
3. Placed on shaker to dissolve all solids.

The following steps were used to prepare 20% SBECD solution in 10 mM citrate, pH 5.5 (300-g scale):
1. Weighed 62.57 g of SBECD into a 1000-mL solvent bottle.
2. Weighed 237.43 g of 10 mM citrate, pH 5.5 into the same bottle.
3. Placed on shaker to dissolve all solids.

The following steps were used to prepare a generic formulation (250-mL scale):
1. Added a stir bar into a 250-mL solvent bottle.
2. Recorded tare weight of bottle with stir bar.
3. Weighed target amount of ABP-700 into same bottle.
4. Weighed 125 mL (˜137.5 g) of SBECD solution in 10 mM citrate buffer into same bottle.
5. Stirred to completely solubilize all API.
6. Transferred 100 mL (˜100 g) of 10 mM citrate buffer into the same bottle.
7. Titrated solution to target pH±0.05 with 0.5N NaOH.
8. QS′d solution to 250-mL with additional 10 mM citrate buffer.

The formulations were tested for stability after storage under various conditions at time points 0, 0.5 months, 1 month, 3 months, 6 months, and 12 months. The formulations were stored in a 10 mL clear type 1 tubing glass vial, which were asceptically filled. Stability was measured using HPLC.

The stability data at 0.5, 1, 2, 3, and 6 months for Formulation 1 (5 mg/mL CPMM, 9.0% SBECD, 10 mM citrate, at pH 5.0) stored at 40° C. under 75% relative humidity is shown in Tables 13 and 14 below.

TABLE 13
0.5- and 1-month stability data for Formulation 1 stored
at 40° C. under 75% relative humidity.

| Parameter | Specification | Initial | 0.5 Month | 1 Month |
|---|---|---|---|---|
| Appearance | Report results | Clear, colorless | Clear, colorless | Clear, colorless |
| | | solution free of | solution free of | solution free of |
| | | particles | particles | particles |
| pH | Report results | 4.99 | 4.93 | 4.95 |
| Potency by | 90.0-110.0% of | 100.7% | 103.0% | 100.6% |
| HPLC | Label Claim | | | |
| Individual | Etomidate Acid | ND | 0.12% | 0.25% |
| Related | RRT 0.45 | | | |
| Substances | RRT 1.17 | 0.22% | 0.22% | 0.23% |
| (Area %) | | | | |
| Total Related | Report the sum of | 0.2% | 0.3% | 0.5% |
| Substances | individual related | | | |
| (Area %) | substances to 1 | | | |
| | decimal place | | | |
| Osmolality | Report results | 338 | NT | NT |
| (mOsm/kg) | | | | |

RH: Relative Humidity
RRT: Relative Retention Time
NT: Not Tested
ND: Not Detected or <0.05% area

TABLE 14
2-, 3-, and 6-month stability data for Formulation 1 stored at 40° C. under
75% relative humidity.

| Parameter | Specification | Initial | 2 Months | 3 Months | 6 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 4.99 | 4.97 | 5.07 | 5.05 |
| Potency by | 90.0-110.0% | 100.7% | 101.7% | 99.4% | 98.8% |
| HPLC | of Label Claim | | | | |
| Individual | Etomidate Acid | ND | 0.63% | 0.95% | 1.87% |
| Related | RRT 0.45 | | | | |
| Substances | CPM Acid | ND | 0.05% | ND | ND |
| (Area %) | RRT 0.76 | | | | |
| | RRT 1.17 | 0.22% | 0.22% | 0.21% | 0.22% |
| Total Related | Report the sum | 0.2% | 0.9% | 1.2% | 2.1% |
| Substances | of individual | | | | |
| (Area %) | related | | | | |
| | substances to 1 | | | | |
| | decimal place | | | | |
| Osmolality | Report results | 338 | NT | NT | NT |
| (mOsm/kg) | | | | | |

The stability data at 3-, 6-, and 12-months for Formulation 1 (5 mg/mL CPMM, 9.0% SBECD, 10 mM citrate, at pH 5.0) stored at 2-8° C. is shown in Table 15 below.

TABLE 15
3-, 6-, and 12-month stability data for Formulation 1 stored at 2-8° C.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 4.99 | 4.99 | 5.05 | 4.98 |
| Potency by | 90.0-110.0% | 100.7% | 101.3% | 100.9% | 102.8% |
| HPLC | of Label Claim | | | | |
| Individual | Etomidate Acid | ND | ND | 0.06% | 0.11% |
| Related | RRT 0.45 | | | | |
| Substances | RRT 1.17 | 0.22% | 0.22% | 0.22% | 0.22% |
| (Area %) | | | | | |
| Total Related | Report the sum | 0.2% | 0.2% | 0.3% | 0.3% |
| Substances | of individual | | | | |
| (Area %) | related | | | | |
| | substances to 1 | | | | |
| | decimal place | | | | |
| Osmolality | Report results | 338 | NT | NT | 319 |
| (mOsm/kg) | | | | | |

The stability data at 3-, 6-, and 12-months for Formulation 1 (5 mg/mL CPMM, 9.0% SBECD, 10 mM citrate, at pH 5.0) stored at 25° C., 60% relative humidity is shown in Table 16 below.

TABLE 16
3-, 6-, and 12-month stability data for Formulation 1 stored at 25° C., 60%
relative humidity.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 4.99 | 5.01 | 5.06 | 4.98 |
| Potency by | 90.0-110.0% | 100.7% | 101.0% | 100.8% | 102.2% |
| HPLC | of Label Claim | | | | |
| Individual | Etomidate Acid | ND | 0.21% | 0.41% | 0.77% |
| Related | RRT 0.45 | | | | |
| Substances | RRT 1.17 | 0.22% | 0.22% | 0.21% | 0.22% |
| (Area %) | | | | | |
| Total Related | Report the sum | 0.2% | 0.4% | 0.6% | 1.0% |
| Substances | of individual | | | | |
| (Area %) | related | | | | |
| | substances to 1 | | | | |
| | decimal place | | | | |
| Osmolality | Report results | 338 | NT | NT | 314 |
| (mOsm/kg) | | | | | |

The stability data at 0.5, 1, 2, 3, and 6 months for Formulation 2 (3 mg/mL CPMM, 9.0% SBECD, 10 mM citrate, at pH 5.0) stored at 40° C. under 75% relative humidity is shown in Tables 17 and 18 below.

TABLE 17
0.5- and 1-month stability data for Formulation 2 stored at 40° C. under 75%
relative humidity.

| Parameter | Specification | Initial | 0.5 Month | 1 Month |
|---|---|---|---|---|
| Appearance | Report results | Clear, colorless | Clear, colorless | Clear, colorless |
| | | solution free of | solution free of | solution free of |
| | | particles | particles | particles |
| pH | Report results | 4.98 | 4.92 | 4.96 |
| Potency by | 90.0-110.0% | 101.1% | 103.5% | 102.3% |
| HPLC | of Label Claim | | | |
| Individual | Etomidate Acid | ND | 0.11% | 0.22% |
| Related | RRT 0.45 | | | |
| Substances | RRT 1.17 | 0.22% | 0.22% | 0.23% |
| (Area %) | | | | |
| Total Related | Report the sum | 0.2% | 0.3% | 0.4% |
| Substances | of individual | | | |
| (Area %) | related | | | |
| | substances to 1 | | | |
| | decimal place | | | |
| Osmolality | Report results | 316 | NT | NT |
| (mOsm/kg) | | | | |

TABLE 18
2-, 3, and 6-month stability data for Formulation 2 stored at 40° C. under 75%
relative humidity.

| Parameter | Specification | Initial | 2 Months | 3 Months | 6 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 4.98 | 4.95 | 5.03 | 5.06 |
| Potency by | 90.0-110.0% | 101.1% | 102.3% | 99.5% | 98.9% |
| HPLC | of Label Claim | | | | |
| Individual | Etomidate Acid | ND | 0.55% | 0.85% | 1.66% |
| Related | RRT 0.45 | | | | |
| Substances | RRT 1.17 | 0.22% | 0.22% | 0.22% | 0.22% |
| (Area %) | | | | | |
| Total Related | Report the sum | 0.2% | 0.8% | 1.1% | 1.9% |
| Substances | of individual | | | | |
| (Area %) | related | | | | |
| | substances to 1 | | | | |
| | decimal place | | | | |
| Osmolality | Report results | 316 | NT | NT | NT |
| (mOsm/kg) | | | | | |

The stability data at 3-, 6-, and 12-months for Formulation 2 (3 mg/mL CPMM, 9.0% SBECD, 10 mM citrate, at pH 5.0) stored at 2-8° C. is shown in Table 19 below.

TABLE 19
3-, 6-, and 12-month stability data for Formulation 2 stored at 2-8° C.

| Parameter | Specification | Initial | 2 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 4.98 | 5.01 | 5.03 | 4.99 |
| Potency by | 90.0-110.0% | 101.1% | 101.1% | 101.1% | 103.9% |
| HPLC | of Label Claim | | | | |
| Individual | Etomidate Acid | ND | ND | 0.05% | 0.09% |
| Related | RRT 0.45 | | | | |
| Substances | RRT 1.17 | 0.22% | 0.22% | 0.22% | 0.22% |
| (Area %) | | | | | |
| Total Related | Report the sum | 0.2% | 0.2% | 0.3% | 0.3% |
| Substances | of individual | | | | |
| (Area %) | related | | | | |
| | substances to 1 | | | | |
| | decimal place | | | | |
| Osmolality | Report results | 316 | NT | NT | 305 |
| (mOsm/kg) | | | | | |

The stability data at 3-, 6-, and 12-months for Formulation 2 (3 mg/mL CPMM, 9.0% SBECD, 10 mM citrate, at pH 5.0) stored at 25° C., 60% relative humidity is shown in Table 20 below.

TABLE 20
3-, 6-, and 12-month stability data for Formulation 2 stored at 25° C., 60%
relative humidity.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 4.98 | 5.07 | 5.06 | 4.99 |
| Potency by | 90.0-110.0% | 101.1% | 100.9% | 101.1% | 102.8% |
| HPLC | of Label Claim | | | | |
| Individual | Etomidate Acid | ND | 0.19% | 0.38% | 0.68% |
| Related | RRT 0.45 | | | | |
| Substances | RRT 1.17 | 0.22% | 0.23% | 0.22% | 0.22% |
| (Area %) | | | | | |
| Total Related | Report the sum | 0.2% | 0.4% | 0.6% | 0.9% |
| Substances | of individual | | | | |
| (Area %) | related | | | | |
| | substances to 1 | | | | |
| | decimal place | | | | |
| Osmolality | Report results | 316 | NT | NT | 398 |
| (mOsm/kg) | | | | | |

The stability data at 0.5, 1, 2, 3, and 6 months for Formulation 3 (5 mg/mL CPMM, 11.0% SBECD, 10 mM citrate, at pH 6.0) stored at 40° C. under 75% relative humidity is shown in Tables 21 and 22 below.

TABLE 21
0.5- and 1-month stability data for Formulation 3 stored at 40° C. under 75%
relative humidity.

| Parameter | Specification | Initial | 0.5 Month | 1 Month |
|---|---|---|---|---|
| Appearance | Report results | Clear, colorless | Clear, colorless | Clear, colorless |
| | | solution free of | solution free of | solution free of |
| | | particles | particles | particles |
| pH | Report results | 5.92 | 5.89 | 5.96 |
| Potency by | 90.0-110.0% | 100.3% | 102.3% | 100.6% |
| HPLC | of Label Claim | | | |
| Individual | Etomidate Acid | ND | 0.08% | 0.17% |
| Related | RRT 0.45 | | | |
| Substances | CPM Acid | ND | 0.09% | 0.16% |
| (Area %) | RRT 0.76 | | | |
| | RRT 1.17 | 0.23% | 0.22% | 0.24% |
| Total Related | Report the sum | 0.2% | 0.4% | 0.6% |
| Substances | of individual | | | |
| (Area %) | related | | | |
| | substances to 1 | | | |
| | decimal place | | | |
| Osmolality | Report results | 411 | NT | NT |
| (mOsm/kg) | | | | |

TABLE 22
2-, 3-, and 6-month stability data for Formulation 3 stored at 40° C. under
75% relative humidity.

| Parameter | Specification | Initial | 2 Months | 3 Months | 6 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.92 | 5.94 | 6.01 | 5.98 |
| Potency by | 90.0-110.0% | 100.3% | 101.5% | 99.4% | 97.0% |
| HPLC | of Label Claim | | | | |
| Individual | Etomidate Acid | ND | 0.41% | 0.65% | 1.34% |
| Related | RRT 0.45 | | | | |
| Substances | CPM Acid | ND | 0.23% | 0.33% | 0.43% |
| (Area %) | RRT 0.76 | | | | |
| | RRT 1.17 | 0.23% | 0.22% | 0.22% | 0.22% |
| Total Related | Report the sum | 0.2% | 0.9% | 1.2% | 2.0% |
| Substances | of individual | | | | |
| (Area %) | related | | | | |
| | substances to 1 | | | | |
| | decimal place | | | | |
| Osmolality | Report results | 411 | NT | NT | NT |
| (mOsm/kg) | | | | | |

The stability data at 3-, 6-, and 12-months for Formulation 3 (5 mg/mL CPMM, 11.0% SBECD, 10 mM citrate, at pH 6.0) stored at 2-8° C. is shown in Table 23 below.

TABLE 23
3-, 6-, and 12-month stability data for Formulation 3 stored at 2-8° C.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.92 | 6.04 | 5.95 | 5.96 |
| Potency by | 90.0-110.0% | 100.3% | 100.0% | 100.3% | 102.9% |
| HPLC | of Label Claim | | | | |
| Individual | Etomidate Acid | ND | ND | 0.05% | 0.06% |
| Related | RRT 0.45 | | | | |
| Substances | RRT 1.17 | 0.23% | 0.22% | 0.22% | 0.22% |
| (Area %) | | | | | |
| Total Related | Report the sum | 0.2% | 0.2% | 0.3% | 0.3% |
| Substances | of individual | | | | |
| (Area %) | related | | | | |
| | substances to 1 | | | | |
| | decimal place | | | | |
| Osmolality | Report results | 411 | NT | NT | 399 |
| (mOsm/kg) | | | | | |

The stability data at 3-, 6-, and 12-months for Formulation 3 (5 mg/mL CPMM, 11.0% SBECD, 10 mM citrate, at pH 6.0) stored at 25° C., 60% relative humidity is shown in Table 24 below.

TABLE 24
3-, 6-, and 12-month stability data for Formulation 3 stored at 25° C., 60%
relative humidity.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.92 | 5.96 | 5.95 | 5.99 |
| Potency by | 90.0-110.0% | 100.3% | 100.2% | 99.7% | 102.4% |
| HPLC | of Label Claim | | | | |
| Individual | Etomidate Acid | ND | 0.12% | 0.19% | 0.37% |
| Related | RRT 0.45 | | | | |
| Substances | RRT 1.17 | 0.23% | 0.22% | 0.22% | 0.22% |
| (Area %) | | | | | |
| Total Related | Report the sum | 0.2% | 0.4% | 0.5% | 0.8% |
| Substances | of individual | | | | |
| (Area %) | related | | | | |
| | substances to 1 | | | | |
| | decimal place | | | | |
| Osmolality | Report results | 411 | NT | NT | 404 |
| (mOsm/kg) | | | | | |

The stability data at 0.5, 1, 2, 3, and 6 months for Formulation 4 (3 mg/mL CPMM, 11.0% SBECD, 10 mM citrate, at pH 5.0) stored at 40° C. under 75% relative humidity is shown in Tables 25 and 26 below.

TABLE 25
0.5- and 1-month stability data for Formulation 4 stored at 40° C. under
75% relative humidity.

| Parameter | Specification | Initial | 0.5 Month | 1 Month |
|---|---|---|---|---|
| Appearance | Report results | Clear, | Clear, | Clear, colorless |
| | | colorless | colorless | solution free of |
| | | solution | solution | particles |
| | | free of | free of | |
| | | particles | particles | |
| pH | Report results | 4.95 | 4.92 | 4.95 |
| Potency by | 90.0-110.0% | 100.2% | 103.0% | 100.9% |
| HPLC | of Label Claim | | | |
| Individual | Etomidate Acid | ND | 0.09% | 0.19% |
| Related | RRT 0.45 | | | |
| Substances | RRT 1.17 | 0.22% | 0.22% | 0.24% |
| (Area %) | | | | |
| Total Related | Report the sum | 0.2% | 0.3% | 0.4% |
| Substances | of individual | | | |
| (Area %) | related | | | |
| | substances to 1 | | | |
| | decimal place | | | |
| Osmolality | Report results | 403 | NT | NT |
| (mOsm/kg) | | | | |

TABLE 26
2-, 3-, and 6-month stability data for Formulation 4 stored at 40° C. under
75% relative humidity.

| Parameter | Specification | Initial | 2 Months | 3 Months | 6 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 4.95 | 4.92 | 5.08 | 5.04 |
| Potency by | 90.0-110.0% | 100.2% | 102.4% | 99.8% | 99.2% |
| HPLC | of Label Claim | | | | |
| Individual | Etomidate Acid | ND | 0.46% | 0.72% | 1.41% |
| Related | RRT 0.45 | | | | |
| Substances | CPM Acid | ND | 0.06% | ND | ND |
| (Area %) | RRT 0.76 | | | | |
| | RRT 1.17 | 0.22% | 0.21% | 0.21% | 0.23% |
| Total Related | Report the sum | 0.2% | 0.7% | 0.9% | 1.6% |
| Substances | of individual | | | | |
| (Area %) | related | | | | |
| | substances to 1 | | | | |
| | decimal place | | | | |
| Osmolality | Report results | 403 | NT | NT | NT |
| (mOsm/kg) | | | | | |

The stability data at 3-, 6-, and 12-months for Formulation 4 (3 mg/mL CPMM, 11.0% SBECD, 10 mM citrate, at pH 5.0) stored at 2-8° C. is shown in Table 27 below.

TABLE 27
3-, 6-, and 12-month stability data for Formulation 4 stored at 2-8° C.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 4.95 | 5.04 | 5.02 | 4.97 |
| Potency by | 90.0-110.0% | 100.2% | 100.4% | 100.3% | 103.5% |
| HPLC | of Label Claim | | | | |
| Individual | Etomidate Acid | ND | ND | ND | 0.08° C. |
| Related | RRT 0.45 | | | | |
| Substances | RRT 1.17 | 0.22% | 0.22% | 0.22% | 0.22% |
| (Area %) | | | | | |
| Total Related | Report the sum | 0.2% | 0.2% | 0.2% | 0.3% |
| Substances | of individual | | | | |
| (Area %) | related | | | | |
| | substances to 1 | | | | |
| | decimal place | | | | |
| Osmolality | Report results | 403 | NT | NT | 384 |
| (mOsm/kg) | | | | | |

The stability data at 3-, 6-, and 12-months for Formulation 4 (3 mg/mL CPMM, 11.0% SBECD, 10 mM citrate, at pH 5.0) stored at 25° C., 60% relative humidity is shown in Table 28 below.

TABLE 28
3-, 6-, and 12-month stability data for Formulation 4 stored at 25° C., 60%
relative humidity.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 4.95 | 4.99 | 5.02 | 4.99 |
| Potency by | 90.0-110.0% | 100.2% | 101.0% | 100.6% | 102.0% |
| HPLC | of Label Claim | | | | |
| Individual | Etomidate Acid | ND | 0.16% | 0.31% | 0.57% |
| Related | RRT 0.45 | | | | |
| Substances | RRT 1.17 | 0.22% | 0.22% | 0.22% | 0.22% |
| (Area %) | | | | | |
| Total Related | Report the sum | 0.2% | 0.4% | 0.5% | 0.8% |
| Substances | of individual | | | | |
| (Area %) | related | | | | |
| | substances to 1 | | | | |
| | decimal place | | | | |
| Osmolality | Report results | 403 | NT | NT | 386 |
| (mOsm/kg) | | | | | |

The stability data at 0.5, 1, 2, 3, and 6 months for Formulation 5 (5 mg/mL CPMM, 9.0% SBECD, 10 mM citrate, at pH 6.0) stored at 40° C. under 75% relative humidity is shown in Tables 29 and 30 below.

TABLE 29
0.5- and 1-month stability data for Formulation 5 stored at 40° C. under
75% relative humidity.

| | | | 0.5 | |
|---|---|---|---|---|
| Parameter | Specification | Initial | Months | 1 Months |
| Appearance | Report results | Clear, | Clear, | Clear, colorless |
| | | colorless | colorless | solution free of |
| | | solution | solution | particles |
| | | free of | free of | |
| | | particles | particles | |
| pH | Report results | 5.91 | 5.91 | 5.97 |
| Potency by | 90.0-110.0% | 100.0% | 102.1% | 100.4% |
| HPLC | of Label Claim | | | |
| Individual | Etomidate Acid | ND | 0.10% | 0.19% |
| Related | RRT 0.45 | | | |
| Substances | CPM Acid | ND | 0.09% | 0.15% |
| (Area %) | RRT 0.76 | | | |
| | RRT 1.17 | 0.23% | 0.22% | 0.22% |
| Total Related | Report the sum | 0.2% | 0.4% | 0.6% |
| Substances | of individual | | | |
| (Area %) | related | | | |
| | substances to 1 | | | |
| | decimal place | | | |
| Osmolality | Report results | 347 | NT | NT |
| (mOsm/kg) | | | | |

TABLE 30
2-, 3-, and 6-month stability data for Formulation 5 stored at 40° C. under
75% relative humidity.

| Parameter | Specification | Initial | 2 Months | 3 Months | 6 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.91 | 5.95 | 6.06 | 5.96 |
| Potency by | 90.0-110.0% | 100.0% | 101.4% | 98.4% | 98.2% |
| HPLC | of Label Claim | | | | |
| Individual | Etomidate Acid | ND | 0.49% | 0.78% | 1.64% |
| Related | RRT 0.45 | | | | |
| Substances | CPM Acid | ND | 0.24% | 0.32% | 0.44% |
| (Area %) | RRT 0.76 | | | | |
| | RRT 1.17 | 0.23% | 0.22% | 0.22% | 0.21% |
| Total Related | Report the sum | 0.2% | 1.0% | 1.3% | 2.3% |
| Substances | of individual | | | | |
| (Area %) | related | | | | |
| | substances to 1 | | | | |
| | decimal place | | | | |
| Osmolality | Report results | 347 | NT | NT | NT |
| (mOsm/kg) | | | | | |

The stability data at 3-, 6-, and 12-months for Formulation 5 (5 mg/mL CPMM, 9.0% SBECD, 10 mM citrate, at pH 6.0) stored at 2-8° C. is shown in Table 31 below.

TABLE 31
3-, 6-, and 12-month stability data for Formulation 5 stored at 2-8° C.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.91 | 6.04 | 5.98 | 5.94 |
| Potency by | 90.0-110.0° C. | 100.0% | 100.2% | 100.1% | 101.9% |
| HPLC | of Label Claim | | | | |
| Individual | Etomidate Acid | ND | ND | ND | 0.05% |
| Related | RRT 0.45 | | | | |
| Substances | CPM Acid | ND | ND | ND | 0.05% |
| (Area %) | RRT 0.76 | | | | |
| | RRT 1.17 | 0.23% | 0.22% | 0.22% | 0.22% |
| Total Related | Report the sum | 0.2% | 0.2% | 0.2% | 0.3% |
| Substances | of individual | | | | |
| (Area %) | related | | | | |
| | substances to 1 | | | | |
| | decimal place | | | | |
| Osmolality | Report results | 347 | NT | NT | 322 |
| (mOsm/kg) | | | | | |

The stability data at 3-, 6-, and 12-months for Formulation 5 (5 mg/mL CPMM, 9.0% SBECD, 10 mM citrate, at pH 6.0) stored at 25° C., 60% relative humidity is shown in Table 32 below.

TABLE 32
3-, 6-, and 12-month stability data for Formulation 5 stored at 25° C., 60%
relative humidity.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.91 | 5.94 | 5.97 | 5.96 |
| Potency by | 90.0-110.0% | 100.0% | 100.3% | 99.3% | 101.1% |
| HPLC | of Label Claim | | | | |
| Individual | Etomidate Acid | ND | 0.12% | 0.25% | 0.46% |
| Related | RRT 0.45 | | | | |
| Substances | CPM Acid | ND | 0.08% | 0.12% | 0.22% |
| (Area %) | RRT 0.76 | | | | |
| | RRT 1.17 | 0.23% | 0.22% | 0.21% | 0.21% |
| Total Related | Report the sum | 0.2% | 0.4% | 0.6% | 0.9% |
| Substances | of individual | | | | |
| (Area %) | related | | | | |
| | substances to 1 | | | | |
| | decimal place | | | | |
| Osmolality | Report results | 347 | NT | NT | 322 |
| (mOsm/kg) | | | | | |

The stability data at 0.5, 1, 2, 3, and 6 months for Formulation 6 (4 mg/mL CPMM, 10.0% SBECD, 10 mM citrate, at pH 5.5) stored at 40° C. under 75% relative humidity is shown in Tables 33 and 34 below.

TABLE 33
0.5-, and 1-month stability data for Formulation 6 stored at 40° C. under
75% relative humidity.

| Parameter | Specification | Initial | 0.5 Month | 1 Month |
|---|---|---|---|---|
| Appearance | Report results | Clear, | Clear, | Clear, |
| | | colorless | colorless | colorless |
| | | solution | solution | solution free |
| | | free of | free of | of particles |
| | | particles | particles | |
| pH | Report results | 5.43 | 5.42 | 5.48 |
| Potency by | 90.0-110.0% of | 100.0% | 103.2% | 101.4% |
| HPLC | Label Claim | | | |
| Individual | Etomidate Acid | ND | 0.07% | 0.15% |
| Related | RRT 0.45 | | | |
| Substances | CPM Acid RRT | ND | ND | 0.06% |
| (Area %) | 0.76 | | | |
| | RRT 1.17 | 0.22% | 0.22% | 0.23% |
| Total Related | Report the sum | 0.2% | 0.3% | 0.4% |
| Substances | of individual | | | |
| (Area %) | related | | | |
| | substances to 1 | | | |
| | decimal place | | | |
| Osmolality | Report results | 356 | NT | NT |
| (mOsm/kg) | | | | |

TABLE 34
2-, 3-, and 6-month stability data for Formulation 6 stored at 40° C. under
75% relative humidity.

| Parameter | Specification | Initial | 2 Months | 3 Months | 6 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.43 | 5.44 | 5.60 | 5.51 |
| Potency by | 90.0-110.0% of | 100.0% | 103.4% | 99.9% | 99.7% |
| HPLC | Label Claim | | | | |
| Individual | Etomidate Acid | ND | 0.39% | 0.59% | 1.17% |
| Related | RRT 0.45 | | | | |
| Substances | CPM Acid RRT | ND | 0.10% | 0.09% | 0.13% |
| (Area %) | 0.76 | | | | |
| | RRT 0.97 | ND | ND | ND | 0.06% |
| | RRT 1.17 | 0.22% | 0.21% | 0.23% | 0.22% |
| Total Related | Report the sum | 0.2% | 0.7% | 0.9% | 1.6% |
| Substances | of individual | | | | |
| (Area %) | related | | | | |
| | substances to 1 | | | | |
| | decimal place | | | | |
| Osmolality | Report results | 356 | NT | NT | NT |
| (mOsm/kg) | | | | | |

The stability data at 3 months for Formulation 6 (4 mg/mL CPMM, 10.0% SBECD, 10 mM citrate, at pH 5.5) stored at 25° C. under ambient relative humidity is shown in Table 35 below.

TABLE 35
3-month stability data for Formulation 6 stored at 25° C. under ambient
relative humidity.

| Parameter | Specification | Initial | 3 Months |
|---|---|---|---|
| Appearance | Report results | Clear, colorless | Clear, colorless |
| | | solution free of | solution free of |
| | | particles | particles |
| pH | Report results | 5.43 | 5.49 |
| Potency by | 90.0-110.0% | 100.0% | 101.0% |
| HPLC | of Label Claim | | |
| Individual | Etomidate Acid | ND | 0.11% |
| Related | RRT 0.45 | | |
| Substances | RRT 1.17 | 0.22% | 0.22% |
| (Area %) | | | |
| Total Related | Report the sum | 0.2% | 0.3% |
| Substances | of individual | | |
| (Area %) | related | | |
| | substances to 1 | | |
| | decimal place | | |
| Osmolality | Report results | 356 | NT |
| (mOsm/kg) | | | |

The stability data at 0.5 and 1 month for Formulation 6 (4 mg/mL CPMM, 10.0% SBECD, 10 mM citrate, at pH 5.5) stored at 60° C. under ambient relative humidity is shown in Table 36 below.

TABLE 36
0.5- and 1-month stability data for Formulation 6 stored at 60° C. under
ambient relative humidity.

| | | | | 1 Month | |
|---|---|---|---|---|---|
| Parameter | Specification | Initial | 0.5 Months | No Sonication | Sonication |
| Appearance | Report results | Clear, colorless | NT | Clear, colorless | Clear, colorless |
| | | solution free of | | solution free of | solution free of |
| | | particles | | particles | particles |
| pH | Report results | 5.43 | NT | NT | NT |
| Potency by | 90.0-110.0% | 100.0% | NT | 99.0% | 100.3% |
| HPLC | of Label Claim | | | | |
| Individual | Etomidate Acid | ND | NT | 1.83% | 1.83% |
| Related | RRT 0.45 | | | | |
| Substances | CPM Acid | ND | NT | 0.20% | 0.20% |
| (Area %) | RRT 0.76 | | | | |
| | RRT 1.17 | 0.22% | NT | 0.23% | 0.22% |
| Total Related | Report the sum | 0.2% | NT | 2.3% | 2.2% |
| Substances | of individual | | | | |
| (Area %) | related | | | | |
| | substances to 1 | | | | |
| | decimal place | | | | |
| Osmolality | Report results | 356 | NT | NT | NT |
| (mOsm/kg) | | | | | |

The stability data at 3-, 6-, and 12-months for Formulation 6 (4 mg/mL CPMM, 10.0% SBECD, 10 mM citrate, at pH 5.5) stored at 2-8° C. is shown in Table 37 below.

TABLE 37
3-, 6-, and 12-month stability data for Formulation 6 stored at 2-8° C.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.43 | 5.47 | 5.52 | 5.50 |
| Potency by | 90.0-110.0% | 100.0% | 100.6% | 101.3% | 103.4% |
| HPLC | of Label Claim | | | | |
| Individual | Etomidate Acid | ND | ND | ND | 0.05% |
| Related | RRT 0.45 | | | | |
| Substances | RRT 1.17 | 0.22% | 0.21% | 0.21% | 0.22% |
| (Area %) | | | | | |
| Total Related | Report the sum | 0.2% | 0.2% | 0.2% | 0.3% |
| Substances | of individual | | | | |
| (Area %) | related | | | | |
| | substances to 1 | | | | |
| | decimal place | | | | |
| Osmolality | Report results | 356 | NT | NT | 349 |
| (mOsm/kg) | | | | | |

The stability data at 3-, 6-, and 12-months for Formulation 6 (4 mg/mL CPMM, 10.0% SBECD, 10 mM citrate, at pH 5.5) stored at 25° C., 60% relative humidity is shown in Table 38 below.

TABLE 38
3-, 6-, and 12-month stability data for Formulation 6 stored at 25° C., 60%
relative humidity.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.43 | 5.49 | 5.51 | 5.49 |
| Potency by | 90.0-110.0% | 100.0% | 101.0% | 101.0% | 102.4% |
| HPLC | of Label Claim | | | | |
| Individual | Etomidate Acid | ND | 0.11% | 0.22% | 0.41% |
| Related | RRT 0.45 | | | | |
| Substances | CPM Acid | ND | ND | 0.05% | 0.07% |
| (Area %) | RRT 0.76 | | | | |
| | RRT 1.17 | 0.22% | 0.22% | 0.22% | 0.22% |
| Total Related | Report the sum | 0.2% | 0.3% | 0.5% | 0.7% |
| Substances | of individual | | | | |
| (Area %) | related | | | | |
| | substances to 1 | | | | |
| | decimal place | | | | |
| Osmolality | Report results | 356 | NT | NT | 348 |
| (mOsm/kg) | | | | | |

The stability data at 0.5, 1, 2, 3, and 6 month for Formulation 7 (3 mg/mL CPMM, 9.0% SBECD, 10 mM citrate, at pH 6.0) stored at 40° C. under 75% relative humidity is shown in Tables 39 and 40 below.

TABLE 39
0.5- and 1-month stability data for Formulation 7 stored at 40° C. under
75% relative humidity.

| Parameter | Specification | Initial | 0.5 Month | 1 Month |
|---|---|---|---|---|
| Appearance | Report results | Clear, | Clear, | Clear, colorless |
| | | colorless | colorless | solution free of |
| | | solution | solution | particles |
| | | free of | free of | |
| | | particles | particles | |
| pH | Report results | 5.92 | 5.90 | 5.95 |
| Potency by | 90.0-110.0% | 101.1% | 102.8% | 101.5% |
| HPLC | of Label Claim | | | |
| Individual | Etomidate Acid | ND | 0.08% | 0.18% |
| Related | RRT 0.45 | | | |
| Substances | CPM Acid | ND | 0.08% | 0.14% |
| (Area %) | RRT 0.76 | | | |
| | RRT 1.17 | 0.23% | 0.22% | 0.23% |
| Total Related | Report the sum | 0.2% | 0.4% | 0.6% |
| Substances | of individual | | | |
| (Area %) | related | | | |
| | substances to 1 | | | |
| | decimal place | | | |
| Osmolality | Report results | 322 | NT | NT |
| (mOsm/kg) | | | | |

TABLE 40
2-, 3-, and 6-month stability data for Formulation 7 stored at 40° C. under 75%
relative humidity.

| Parameter | Specification | Initial | 2 Months | 3 Months | 6 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.92 | 5.93 | 6.09 | 5.96 |
| Potency by | 90.0-110.0% | 101.1% | 103.0% | 99.8% | 99.1% |
| HPLC | of Label Claim | | | | |
| Individual | Etomidate Acid | ND | 0.41% | 0.69% | 1.49% |
| Related | RRT 0.45 | | | | |
| Substances | CPM Acid | ND | 0.22% | 0.30% | 0.44% |
| (Area %) | RRT 0.76 | | | | |
| | RRT 1.17 | 0.23% | 0.22% | 0.22% | 0.22% |
| Total Related | Report the sum | 0.2% | 0.8% | 1.2% | 2.1% |
| Substances | of individual | | | | |
| (Area %) | related | | | | |
| | substances to 1 | | | | |
| | decimal place | | | | |
| Osmolality | Report results | 322 | NT | NT | NT |
| (mOsm/kg) | | | | | |

The stability data at 3-, 6-, and 12-months for Formulation 7 (3 mg/mL CPMM, 9.0% SBECD, 10 mM citrate, at pH 6.0) stored at 2-8° C. is shown in Table 41 below.

TABLE 41
3-, 6-, and 12-month stability data for Formulation 7 stored at 2-8° C.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.92 | 5.96 | 5.98 | 5.97 |
| Potency by | 90.0-110.0% | 101.1% | 100.4% | 101.5% | 102.9% |
| HPLC | of Label Claim | | | | |
| Individual | RRT 1.17 | 0.23% | 0.22% | 0.21% | 0.22% |
| Related | | | | | |
| Substances | | | | | |
| (Area %) | | | | | |
| Total Related | Report the sum | 0.2% | 0.2% | 0.2% | 0.2% |
| Substances | of individual | | | | |
| (Area %) | related | | | | |
| | substances to 1 | | | | |
| | decimal place | | | | |
| Osmolality | Report results | 322 | NT | NT | 311 |
| (mOsm/kg) | | | | | |

The stability data at 3-, 6-, and 12-months for Formulation 7 (3 mg/mL CPMM, 9.0% SBECD, 10 mM citrate, at pH 6.0) stored at 25° C., 60% relative humidity is shown in Table 42 below.

TABLE 42
3-, 6-, and 12-month stability data for Formulation 7 stored at 25° C., 60%
relative humidity.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.92 | 5.96 | 5.98 | 5.97 |
| Potency by | 90.0-110.0% | 101.1% | 100.5% | 101.6% | 102.9% |
| HPLC | of Label Claim | | | | |
| Individual | Etomidate Acid | ND | 0.10% | 0.21% | 0.40% |
| Related | RRT 0.45 | | | | |
| Substances | CPM Acid | ND | 0.07% | 0.12% | 0.20% |
| (Area %) | RRT 0.76 | | | | |
| | RRT 1.17 | 0.23% | 0.22% | 0.21% | 0.21% |
| Total Related | Report the sum | 0.2% | 0.4% | 0.5% | 0.8% |
| Substances | of individual | | | | |
| (Area %) | related | | | | |
| | substances to 1 | | | | |
| | decimal place | | | | |
| Osmolality | Report results | 322 | NT | NT | 322 |
| (mOsm/kg) | | | | | |

The stability data at 0.5, 1, 2, 3, and 6 months for Formulation 8 (4 mg/mL CPMM, 10.0% SBECD, 10 mM citrate, at pH 5.5) stored at 40° C. under 75% relative humidity is shown in Tables 43 and 44 below.

TABLE 43
0.5- and 1-month stability data for Formulation 8 stored at 40° C. under 75%
relative humidity.

| Parameter | Specification | Initial | 0.5 Month | 1 Month |
|---|---|---|---|---|
| Appearance | Report results | Clear, colorless | Clear, colorless | SClear, colorless |
| | | solution free of | solution free of | solution free of |
| | | particles | particles | particles |
| pH | Report results | 5.43 | 5.43 | 5.46 |
| Potency by | 90.0-110.0% of | 101.3% | 103.0% | 101.9% |
| HPLC | Label Claim | | | |
| Individual | Etomidate Acid | ND | 0.07% | 0.15% |
| Related | RRT 0.45 | | | |
| Substances | CPM Acid RRT | ND | ND | 0.06% |
| (Area %) | 0.76 | | | |
| | RRT 1.17 | 0.22% | 0.22% | 0.22% |
| Total Related | Report the sum | 0.2% | 0.3% | 0.4% |
| Substances | of individual | | | |
| (Area %) | related | | | |
| | substances to 1 | | | |
| | decimal place | | | |
| Osmolality | Report results | 370 | NT | NT |
| (mOsm/kg) | | | | |

TABLE 44
2-, 3-, and 6-month stability data for Formulation 8 stored at 40° C. under 75%
relative humidity.

| Parameter | Specification | Initial | 2 Month | 3 Month | 2 Month |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.43 | 5.46 | 5.47 | 5.50 |
| Potency by | 90.0-110.0% of | 101.3% | 103.2% | 100.0% | 100.9% |
| HPLC | Label Claim | | | | |
| Individual | Etomidate Acid | ND | 0.38% | 0.60% | 1.18% |
| Related | RRT 0.45 | | | | |
| Substances | CPM Acid RRT | ND | 0.08% | 0.10% | 0.12% |
| (Area %) | 0.76 | | | | |
| | RRT 1.17 | 0.22% | 0.22% | 0.22% | 0.23% |
| Total Related | Report the sum | 0.2% | 0.7% | 0.9% | 1.5% |
| Substances | of individual | | | | |
| (Area %) | related | | | | |
| | substances to 1 | | | | |
| | decimal place | | | | |
| Osmolality | Report results | 370 | NT | NT | NT |
| (mOsm/kg) | | | | | |

The stability data at 3-, 6-, and 12-months for Formulation 8 (4 mg/mL CPMM, 10.0% SBECD, 10 mM citrate, at pH 5.5) stored at 2-8° C. is shown in Table 45 below.

TABLE 45
3-, 6-, and 12-month stability data for Formulation 8 stored at 2-8° C.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.43 | 5.48 | 5.50 | 5.48 |
| Potency by | 90.0-110.0% | 101.3% | 100.8% | 102.2% | 102.5% |
| HPLC | of Label Claim | | | | |
| Individual | Etomidate Acid | ND | ND | ND | 0.05% |
| Related | RRT 0.45 | | | | |
| Substances | RRT 1.17 | 0.22% | 0.22% | 0.22% | 0.22% |
| (Area %) | | | | | |
| Total Related | Report the sum | 0.2% | 0.2% | 0.2% | 0.3% |
| Substances | of individual | | | | |
| (Area %) | related | | | | |
| | substances to 1 | | | | |
| | decimal place | | | | |
| Osmolality | Report results | 370 | NT | NT | 356 |
| (mOsm/kg) | | | | | |

The stability data at 3-, 6-, and 12-months for Formulation 8 (4 mg/mL CPMM, 10.0% SBECD, 10 mM citrate, at pH 5.5) stored at 25° C. under 60% relative humidity is shown in Table 46 below.

TABLE 46
3-, 6-, and 12-month stability data for Formulation 8 stored at 25° C. under
60% relative humidity.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.43 | 5.53 | 5.52 | 5.49 |
| Potency by | 90.0-110.0% | 101.3% | 100.6% | 101.3% | 102.3% |
| HPLC | of Label Claim | | | | |
| Individual | Etomidate Acid | ND | 0.13% | 0.22% | 0.41% |
| Related | RRT 0.45 | | | | |
| Substances | CPM Acid | ND | ND | 0.05% | 0.06% |
| (Area %) | RRT 0.76 | | | | |
| | RRT 1.17 | 0.22% | 0.22% | 0.21% | 0.22% |
| Total Related | Report the sum | 0.2% | 0.3% | 0.5% | 0.7% |
| Substances | of individual | | | | |
| (Area %) | related | | | | |
| | substances to 1 | | | | |
| | decimal place | | | | |
| Osmolality | Report results | 370 | NT | NT | 349 |
| (mOsm/kg) | | | | | |

The stability data at 0.5, 1, 2, 3, and 6 months for Formulation 9 (3 mg/mL CPMM, 11.0% SBECD, 10 mM citrate, at pH 6.0) stored at 40° C. under 75% relative humidity is shown in Tables 47 and 48 below.

TABLE 47
0.5- and 1-month stability data for Formulation 9 stored at
40° C. under 75% relative humidity.

| Parameter | Specification | Initial | 0.5 Month | 1 Month |
|---|---|---|---|---|
| Appearance | Report results | Clear, colorless | Clear, | Clear, |
| | | solution free of | colorless | colorless |
| | | particles | solution | solution |
| | | | free of | free of |
| | | | particles | particles |
| pH | Report results | 5.88 | 5.88 | 5.94 |
| Potency by | 90.0-110.0% | 101.2% | 103.1% | 101.5% |
| HPLC | of Label Claim | | | |
| Individual | Etomidate Acid | ND | 0.06% | 0.14% |
| Related | RRT 0.45 | | | |
| Substances | CPM Acid | ND | 0.08% | 0.12% |
| (Area %) | RRT 0.76 | | | |
| | RRT 1.17 | 0.22% | 0.22% | 0.23% |
| Total Related | Report the sum | 0.2% | 0.4% | 0.5% |
| Substances | of individual | | | |
| (Area %) | related | | | |
| | substances to 1 | | | |
| | decimal place | | | |
| Osmolality | Report results | 405 | NT | NT |
| (mOsm/kg) | | | | |

TABLE 48
2-, 3-, and 6-month stability data for Formulation 9 stored at 40° C. under 75%
relative humidity.

| Parameter | Specification | Initial | 2 Months | 3 Months | 6 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.88 | 5.93 | 6.08 | 6.03 |
| Potency by | 90.0-110.0% | 101.2% | 103.0% | 99.5% | 99.3% |
| HPLC | of Label Claim | | | | |
| Individual | Etomidate Acid | ND | 0.35% | 0.55% | 1.23% |
| Related | RRT 0.45 | | | | |
| Substances | CPM Acid | ND | 0.20% | 0.27% | 0.41% |
| (Area %) | RRT 0.76 | | | | |
| | RRT 1.17 | 0.22% | 0.22% | 0.22% | 0.21% |
| Total Related | Report the sum | 0.2% | 0.8% | 1.0% | 1.9% |
| Substances | of individual | | | | |
| (Area %) | related | | | | |
| | substances to 1 | | | | |
| | decimal place | | | | |
| Osmolality | Report results | 405 | NT | NT | NT |
| (mOsm/kg) | | | | | |

The stability data at 3-, 6-, and 12-months for Formulation 9 (3 mg/mL CPMM, 11.0% SBECD, 10 mM citrate, at pH 6.0) stored at 2-8° C. is shown in Table 49 below.

TABLE 49
3-, 6-, and 12-month stability data for Formulation 9 stored at 2-8° C.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.88 | 6.02 | 5.97 | 5.95 |
| Potency by | 90.0-110.0% | 101.2% | 100.2% | 102.3% | 103.6% |
| HPLC | of Label Claim | | | | |
| Individual | RRT 1.17 | 0.22% | 0.22% | 0.21% | 0.22% |
| Related | | | | | |
| Substances | | | | | |
| (Area %) | | | | | |
| Total Related | Report the sum | 0.2% | 0.2% | 0.2% | 0.2% |
| Substances | of individual | | | | |
| (Area %) | related | | | | |
| | substances to 1 | | | | |
| | decimal place | | | | |
| Osmolality | Report results | 405 | NT | NT | 387 |
| (mOsm/kg) | | | | | |

The stability data at 3-, 6-, and 12-months for Formulation 9 (3 mg/mL CPMM, 11.0% SBECD, 10 mM citrate, at pH 6.0) stored at 25° C. under 60% relative humidity is shown in Table 50 below.

TABLE 50
3-, 6-, and 12-month stability data for Formulation 9 stored at 25° C. under
60% relative humidity.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 5.88 | 5.97 | 5.97 | 5.99 |
| Potency by | 90.0-110.0% | 101.2% | 101.4% | 101.5% | 102.3% |
| HPLC | of Label Claim | | | | |
| Individual | Etomidate Acid | ND | 0.09% | 0.17% | 0.33% |
| Related | RRT 0.45 | | | | |
| Substances | CPM Acid | ND | 0.07% | 0.12% | 0.19% |
| (Area %) | RRT 0.76 | | | | |
| | RRT 1.17 | 0.22% | 0.22% | 0.22% | 0.22% |
| Total Related | Report the sum | 0.2% | 0.4% | 0.5% | 0.7% |
| Substances | of individual | | | | |
| (Area %) | related | | | | |
| | substances to 1 | | | | |
| | decimal place | | | | |
| Osmolality | Report results | 405 | NT | NT | 422 |
| (mOsm/kg) | | | | | |

The stability data at 0.5, 1, 2, 3, and 6 months for Formulation 10 (5 mg/mL CPMM, 11.0% SBECD, 10 mM citrate, at pH 5.0) stored at 40° C. under 75% relative humidity is shown in Tables 51 and 52 below.

TABLE 51
0.5- and 1-month stability data for Formulation 10 stored at
40° C. under 75% relative humidity.

| Parameter | Specification | Initial | 0.5 Month | 1 Month |
|---|---|---|---|---|
| Appearance | Report results | Clear, colorless | Clear, | Clear, |
| | | solution free of | colorless | colorless |
| | | particles | solution | solution |
| | | | free of | free of |
| | | | particles | particles |
| pH | Report results | 4.89 | 4.89 | 4.97 |
| Potency by | 90.0-110.0% | 100.6% | 102.8% | 100.9% |
| HPLC | of Label Claim | | | |
| Individual | Etomidate Acid | ND | 0.10% | 0.21% |
| Related | RRT 0.45 | | | |
| Substances | RRT 1.17 | 0.23% | 0.22% | 0.23% |
| (Area %) | | | | |
| Total Related | Report the sum | 0.2% | 0.3% | 0.4% |
| Substances | of individual | | | |
| (Area %) | related | | | |
| | substances to 1 | | | |
| | decimal place | | | |
| Osmolality | Report results | 407 | NT | NT |
| (mOsm/kg) | | | | |

TABLE 52
2-, 3-, and 6-month stability data for Formulation 10 stored at 40° C. under 75%
relative humidity.

| Parameter | Specification | Initial | 2 Months | 3 Months | 6 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 4.89 | 4.90 | 5.06 | 5.04 |
| Potency by | 90.0-110.0% | 100.6% | 101.2% | 99.6% | 99.0% |
| HPLC | of Label Claim | | | | |
| Individual | Etomidate Acid | ND | 0.51% | 0.56% | 1.57% |
| Related | RRT 0.45 | | | | |
| Substances | CPM Acid | ND | ND | 0.27% | ND |
| (Area %) | RRT 0.76 | | | | |
| | RRT 1.17 | 0.23% | 0.25% | 0.21% | 0.21% |
| Total Related | Report the sum | 0.2% | 0.7% | 1.0% | 1.8% |
| Substances | of individual | | | | |
| (Area %) | related | | | | |
| | substances to 1 | | | | |
| | decimal place | | | | |
| Osmolality | Report results | 407 | NT | NT | NT |
| (mOsm/kg) | | | | | |

The stability data at 3-, 6-, and 12-months for Formulation 10 (5 mg/mL CPMM, 11.0% SBECD, 10 mM citrate, at pH 5.0) stored at 2-8° C. is shown in Table 53 below.

TABLE 53
3-, 6-, and 12-month stability data for Formulation 10 stored at 2-8° C.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 4.89 | 4.99 | 5.02 | 4.96 |
| Potency by | 90.0-110.0% | 100.6% | 99.4% | 101.5% | 102.5% |
| HPLC | of Label Claim | | | | |
| Individual | Etomidate Acid | ND | ND | 0.05% | 0.09% |
| Related | RRT 0.45 | | | | |
| Substances | RRT 1.17 | 0.23% | 0.22% | 0.21% | 0.22% |
| (Area %) | | | | | |
| Total Related | Report the sum | 0.2% | 0.2% | 0.3% | 0.3% |
| Substances | of individual | | | | |
| (Area %) | related | | | | |
| | substances to 1 | | | | |
| | decimal place | | | | |
| Osmolality | Report results | 407 | NT | NT | 393 |
| (mOsm/kg) | | | | | |

The stability data at 3-, 6-, and 12-months for Formulation 10 (5 mg/mL CPMM, 11.0% SBECD, 10 mM citrate, at pH 5.0) stored at 25° C. under 60% relative humidity is shown in Table 54 below.

TABLE 54
3-, 6-, and 12-month stability data for Formulation 10 stored at 25° C. under
60% relative humidity.

| Parameter | Specification | Initial | 3 Months | 6 Months | 12 Months |
|---|---|---|---|---|---|
| Appearance | Report results | Clear, colorless | Clear, colorless | Clear, colorless | Clear, colorless |
| | | solution free of | solution free of | solution free of | solution free of |
| | | particles | particles | particles | particles |
| pH | Report results | 4.89 | 5.00 | 5.02 | 4.93 |
| Potency by | 90.0-110.0% | 100.6% | 101.5% | 101.7% | 101.6% |
| HPLC | of Label Claim | | | | |
| Individual | Etomidate Acid | ND | 0.08% | 0.35% | 0.64% |
| Related | RRT 0.45 | | | | |
| Substances | CPM Acid | ND | 0.06% | ND | ND |
| (Area %) | RRT 0.76 | | | | |
| | RRT 1.17 | 0.23% | 0.22% | 0.21% | 0.22% |
| Total Related | Report the sum | 0.2% | 0.4% | 0.6% | 0.9% |
| Substances | of individual | | | | |
| (Area %) | related | | | | |
| | substances to 1 | | | | |
| | decimal place | | | | |
| Osmolality | Report results | 407 | NT | NT | 395 |
| (mOsm/kg) | | | | | |

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

1. A formulation comprising a solubilizer and a compound cyclopropyl-MOC-metomidate (CPMM) having a structure 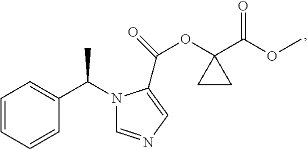 [SMILES: COC(=O)C1(OC(=O)c2cncn2[C@H](C)c2ccccc2)CC1]
or pharmaceutically acceptable salt thereof.

2. The formulation of claim 1, wherein the compound is present in a concentration of 0.1 mg/mL to 20 mg/mL.

3-4. (canceled)

5. The formulation of any one of claim 1 having a pH in a range of 2 to 7.

6-9. (canceled)

10. The formulation of claim 1 further comprising a buffer.

11. The formulation of claim 10, wherein the buffer comprises one or more of NaOH, KOH, triethylamine, meglumine, diethanolamine, triethylamine, ammonium hydroxide, ammonium acetate, L-arginine, histidine, citrate buffer, a phosphate buffer, sodium bicarbonate, tris(hydroxymethyl)aminomethane), N-(2-hydroxyethyl)piperazine-N′-2-ethanesulfonic acid, acetate, citrate, ascorbate, glycine, glutamate, lactate, malate, formate, and sulfate.

12. (canceled)

13. The formulation of claim 1, wherein the solubilizer comprises a cyclodextrin.

14-16. (canceled)

17. The formulation of claim 1, wherein the solubilizer is present in a concentration in a range of about 6% w/w to about 30% w/w.

18-20. (canceled)

21. The formulation of claim 1, wherein the solubilizer is present at a molar ratio to the compound of 1:1 to 8:1.

22. (canceled)

23. The formulation of claim 1, wherein the ratio of compound complexed to the solubilizer to uncomplexed compound is 1.5:1 to 16:1.

24. (canceled)

25. The formulation of claim 1 having 5% or less total degradants after storage at a temperature of 2° C. to 8° C. for at least 6 months.

26-28. (canceled)

29. The formulation of claim 1 having 5% or less total degradants after storage at frozen conditions (e.g., −10° C. to 0° C.) for at least 12 months.

30-31 (canceled)

32. The formulation of claim 1 having 5% or less total degradants after storage at room temperature (e.g., 15 to 30° C.) for at least 6 months.

33. (canceled)

34. The formulation of claim 1, wherein the compound is at a concentration in a range of 2 mg/mL to 5 mg/mL, the solubilizer comprises hydroxypropyl-β-cyclodextrin at a concentration in a range of 8% w/v or w/w to 12% w/v or w/w, and the formulation has a pH in a range of about 5 to 7.

35. The formulation claim 1, wherein the compound is at a concentration in a range of 2 mg/mL to 5 mg/mL, the solubilizer comprises sulfobutylether-β-cyclodextrin at a concentration in a range of 8% w/v or w/w to 12% w/v or w/w, and the formulation has a pH in a range of about 3 to 7.

36. (canceled)

37. A lyophilized pharmaceutical composition made from the formulation of claim 1.

38. A kit comprising the lyophilized formulation of claim 37 in a container and instructions for preparing an aqueous, sterile formulation from the lyophilized formulation and a diluent.

39. (canceled)

40. A liquid pharmaceutical formulation consisting essentially of (1) cyclopropyl-MOC-metomidate (CPMM) or a pharmaceutically acceptable salt thereof, (2) a solubilizer, and (3) a buffer or base in an aqueous medium.

41-44. (canceled)

45. A pre-filled syringe comprising the formulation of claim 1.

46. A method comprising administering to a subject a formulation of claim 1 to induce anesthesia.

47. A method comprising administering to a subject a formulation of claim 1 to induce sedation.
